SEQUENCE LISTING (28 sequences)

SEQ ID NO: 1 - DNA, 20 bp, Artificial Sequence
  source: /note=“Description of Artificial Sequence Synthetic primer”
tggacctgacctgccgtcta

SEQ ID NO: 2 - DNA, 24 bp, Artificial Sequence
  source: /note=“Description of Artificial Sequence Synthetic primer”
ccccagaccctagaataagacagg

SEQ ID NO: 3 - DNA, 25 bp, Artificial Sequence
  source: /note=“Description of Artificial Sequence Synthetic primer”
aacagttgcattatgatatgcccag

SEQ ID NO: 4 - DNA, 21 bp, Artificial Sequence
  source: /note=“Description of Artificial Sequence Synthetic primer”
tgctttcaagcaacttcggga

SEQ ID NO: 5 - DNA, 23 bp, Artificial Sequence
  source: /note=“Description of Artificial Sequence Synthetic primer”
aaaacctgccaaatatgatgaca

SEQ ID NO: 6 - DNA, 20 bp, Artificial Sequence
  source: /note=“Description of Artificial Sequence Synthetic primer”
aagtaccaggcagtgacagc

SEQ ID NO: 7 - RNA, 20 nt, Artificial Sequence
  source: /note=“Description of Artificial Sequence Synthetic oligonucleotide”
cuuccucuugugcucuugcu

SEQ ID NO: 8 - RNA, 20 nt, Artificial Sequence
  source: /note=“Description of Artificial Sequence Synthetic oligonucleotide”
ggaagggcaggcaacgcaug

SEQ ID NO: 9 - DNA, 720 bp, Artificial Sequence
  source: /note=“Description of Artificial Sequence Synthetic polynucleotide”
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 10 - DNA, 66 bp, Artificial Sequence
  source: /note=“Description of Artificial Sequence Synthetic oligonucleotide”
ggaagcggagcgacgaattttagtctactgaaacaagcgggagacgtggaggaaaaccctggacct

SEQ ID NO: 11 - protein, 22 aa, Artificial Sequence
  source: /note=“Description of Artificial Sequence Synthetic peptide”
GSGATNFSLLKQAGDVEENPGP

SEQ ID NO: 12 - DNA, 800 bp, Artificial Sequence
  source: /note=“Description of Artificial Sequence Synthetic polynucleotide”
ttggtatcgtggaaggactcatggtatgagagctggggaatgggactgaggctcccacctttctcatccaagactggctcctccctgccggggctgcgtgcaaccctggggttgggggttctggggactggctttcccataatttcctttcaaggtggggagggaggtagaggggtgatgtggggagtacgctgcagggcctcactccttttgcagaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactcttttcatcttctaggtatgacaacgaatttggctacagcaacagggtggtggacctcatggcccacatggcctccaaagag

SEQ ID NO: 13 - DNA, 800 bp, Artificial Sequence
  source: /note=“Description of Artificial Sequence Synthetic polynucleotide”
ccctggaccaccagccaaagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcaccactacttcagagaacaaggccttttcctctcctcgctccagtcctaggctatctgctgttggccaaacatggaagaagctattctgtgggcagccccagggaggctgacaggtggaggaagtcagggctcgcactgggctctgacgctgactggttagtggagctcagcctggagctgagctgcagcgggcaattccagcttggcctccgcagctgtgaggtcttgagcacgtgctctattgctttctgtgccctcgtgtcttatctgaggacatcgtggccagcccctaaggtcttcaagcaggattcatctaggtaaaccaagtacctaaaaccatgcccaaggcggtaaggactatataatgtttaaaaatcggtaaaaatgcccacctcgcatagtttt

SEQ ID NO: 14 - DNA, 800 bp, Artificial Sequence
  source: /note=“Description of Artificial Sequence Synthetic polynucleotide”
tcttaagcccctctctttctctaacagaaaaagcggatggtggttcctgctgccctcaaggtcgtgcgtctgaagcctacaagaaaggtgagtcccagcttacgctgcaccatctacttgggagatttcaggcctgctgagggacctggggacctggagcctggcagatgatgtccttatctcacgatggtctgcggatgtccctgtgggaatggcgacaatgccaatggcttagctgatgccaggaggcttgggtgggtgcttttctaacaggcctgcagagaacagttgcattatgatatgcccagctgtcagtcacctcccagctctcaacagctccggctcttcagggtgtgggggcttagatatccttacaacttcatttgttcaccccccccccccccccccgcagtttgcctatctggggcgcctggctcacgaggttggctggaagtaccaggcagtgacagccaccctggaggagaagaggaaagagaaagccaagatccactaccggaagaagaaacagctcatggtgaggccaggggctggtgctgaggggggcatctcactcctggacaggcctggcaggtgccttgctcacagagtactcttaactggcaaaggaccagccggggttggggtgggatgcagtccatgtaatgagggcaatgcaacccctcctgaccaccaccacctgcacttattcttggcagaggctacggaaacaggccgagaagaacgtggagaagaaaattgacaaatacacagaggtcctcaagacccacggactcttagtc

SEQ ID NO: 15 - DNA, 800 bp, Artificial Sequence
  source: /note=“Description of Artificial Sequence Synthetic polynucleotide”
gcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaatctggcgtctttgcactgtgtctgctgtggaggcaggcccctggcaaatggggggtgaggagcttgaagagggtagaatgggctgtgctaatatacagaatatatgtaacttgctataaattgaatgatcctttatagacaccgtttacaaaccaaagacataaaatgtggccagcagtgcctggtgcttcctagttaatgtaaagctgtctcattctaattcagctgcaaagtatggacccatgccctgctgccaggctgctgtagtcccggcggtctgtagagactagcattttgcaaatgataa

SEQ ID NO: 16 - DNA, 800 bp, Artificial Sequence
  source: /note=“Description of Artificial Sequence Synthetic polynucleotide”
ctgagctgccaacctggcaattattgtctgctaagggttctctttattcacccttacttggacttcctttcctgtagggaatctcacgtaaaatgaaatcttccctcccccagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtaaaaggatcccaccaggaccacagtgggcctgactgtgacaaattagcagggtgatgtggccttctaccttactgcttttatagttgtattttatatagcagataattttgtgaggggatatttgagaggttgggaggcagggaaggcgtttctcacttgagaaatgacaagagacccaaagagggggttaatgggcaagagctgggccttaggaaccctgcctcactaggccatacccaagctgtcctgcttgggctgcttctgacaggaaaggcttcacacggactttgatattgttggtccttaaactctaccaaggcaggagggtggtgggtaatagaggagtgtggatgaccattttgaccacttcccccctcctttcaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgac

SEQ ID NO: 17 - DNA, 800 bp, Artificial Sequence
  source: /note=“Description of Artificial Sequence Synthetic polynucleotide”
tcaccaaaaagcaacgaacttagccagttttatttgcaaaacaaggaaataaatgcttacttctttaaaaagtctcttgactcttaattttgtaattttttttcctttttgacacagggtctggctgttgcccaggctggagtgtggtggtgtaatcataactcactgcacccttgaactcctgggatcaagggatcctcgtatctcagcctcccaagtagctgggactacaggcacacaccatgacactcagctactaatttttaaattttttttttgtagagatgttgcacaagctggtctcaaattcctggcctcaaggaatcctgcctcagcctcccaaagtgctaggattacaggcttgagccaccatgtgcctggcccttaattttgaggtttatagtgccatatgctagaaacgaaagccatggtaaaaccagagctttgtatttaggtgttgatgtttgggtatctaaatgaagctaccaatcaaacatcctatacagttttctagacacagttgtaactattacactagaattactgtttctatggctgctgcatacttggagtaggtttagtgtcagctgagataggcacctggtggatgctggggccagtcccctagagtaaagtttttcaaactgggtggtgctccaactcggtggtaaccaatttatattttcgagatagtctcaaatatatttgagactggggtgcagtggcttggacttggctcactgcaacctccgcctcctgggttcaagtgattctcctgcctcagcctcccaagtagctgc

SEQ ID NO: 18 - DNA, 684 bp, Artificial Sequence
  source: /note=“Description of Artificial Sequence Synthetic polynucleotide”
atggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccaacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagacctttaa

SEQ ID NO: 19 - protein, 16 aa, Artificial Sequence
  source: /note=“Description of Artificial Sequence Synthetic peptide”
SGGGGSGGGGSGGGGS

SEQ ID NO: 20 - DNA, 819 bp, Artificial Sequence
  source: /note=“Description of Artificial Sequence Synthetic polynucleotide”
atggatatgagagtgcctgcccaacttctcggactgctgctgctttggcttagaggtgcaagatgcgacattgtgctgacacagtctcctgcttccttagctgtatctctgggacagagggccaccatctcatgcagggccagccaaagtgtcagtacatctagatatagttatatacactggtaccaacagaaaccaggacagccacccaaactcctcatcaagtatgcatccaacctagaatctggggtccctgccaggttcagtggcagtgggtctgggacagacttcaccctcaacatccatcctctggaggaggaggatgctgcaacatattactgtcaccacagttgggagattccgctcacgttcggtgctgggaccaagctggagctgaaatccggtggaggcggttcaggcggaggtggctctggcggtggcggatcggaagtgaaggttgaggagtctggaggaggcttggtgcaacctggaggatccatgaaactctcctgtgttgtctctggattcactttcagtaactactgggtgaactgggtccgccagtctccagagaaggggcttgagtgggttgctcaaattagattgaaatctgataattatgcaacacattatgaggagtctgtgaaagggaggttcaccatctcaagagatgattccaaaagtagtgtctatctgcaaatgaacaacctaagggctgaagacagtggaatttattactgcactaactgggaagactactggggccaaggcaccactctcacagtctcctcatacccatacgatgttccagattacgct

SEQ ID NO: 21 - protein, 9 aa, Influenza virus
YPYDVPDYA

SEQ ID NO: 22 - protein, 5 aa, Artificial Sequence
  source: /note=“Description of Artificial Sequence Synthetic peptide”
SGGGS

SEQ ID NO: 23 - DNA, 1179 bp, Artificial Sequence
  source: /note=“Description of Artificial Sequence Synthetic polynucleotide”
atggcttctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagctcgagtatagcggcggcggcagcggcgtggatggcttcggcgacgtgggagccctggagagcctgagaggcaacgccgatctggcctacatcctgagcatggagccctgtggccactgcctgatcatcaacaacgtgaacttctgccgggagagcggcctgcggacccggaccggcagcaacatcgactgcgagaagctgaggaggcgcttctcctccctgcactttatggtggaggtgaaaggcgatctgactgccaagaaaatggtgctggccctgctggagctggcccagcaggaccacggagccctggattgctgtgtggtggtgatcctgtcccacggctgccaggccagccacctgcagttccccggagccgtgtacggcaccgacggctgtcccgtgtccgtggagaagatcgtgaacatcttcaacggcacctcctgcccctccctgggcggcaagcccaagctgttctttatccaggcctgtggcggcgagcagaaggaccacggctttgaggtggccagcacctcccccgaggacgagagcccaggcagcaaccccgagcccgacgccacccccttccaggagggcctgcgcaccttcgaccagctggacgccatcagcagcctgcccacccccagcgacatcttcgtgagctacagcacctttcccggcttcgtgagctggcgcgatcccaagtccggctcttggtatgtggagaccctggacgacatctttgagcagtgggctcatagcgaggacctgcagagcctgctgctgcgcgtggccaatgccgtgagcgtgaagggcatctacaagcagatgccaggctgcttcaacttcctgcggaagaagctgttcttcaagaccagcgcctcctga

SEQ ID NO: 24 - DNA, 1209 bp, Artificial Sequence
  source: /note=“Description of Artificial Sequence Synthetic polynucleotide”
atgctggagggcgtgcaggtggagaccatcagcccaggcgacggcagaaccttccccaagagaggccagacctgcgtggtgcactataccggcatgctggaggacggcaagaagttcgacagcagccgcgaccgcaataagcccttcaagttcatgctgggcaagcaggaggtgatcagaggctgggaggagggcgtggcccagatgagcgtgggccagagagccaagctgaccatcagccccgactacgcctatggcgccaccggccaccccggcatcatcccaccccacgccaccctggtgtttgatgtggagctgctgaagctggagtccggaggcggctccggcgtggatggcttcggcgacgtgggagccctggagagcctgagaggcaacgccgatctggcctacatcctgagcatggagccctgtggccactgcctgatcatcaacaacgtgaacttctgccgggagagcggcctgcggacccggaccggcagcaacatcgactgcgagaagctgaggaggcgcttctcctccctgcactttatggtggaggtgaaaggcgatctgactgccaagaaaatggtgctggccctgctggagctggcccagcaggaccacggagccctggattgctgtgtggtggtgatcctgtcccacggctgccaggccagccacctgcagttccccggagccgtgtacggcaccgacggctgtcccgtgtccgtggagaagatcgtgaacatcttcaacggcacctcctgcccctccctgggcggcaagcccaagctgttctttatccaggcctgtggcggcgagcagaaggaccacggctttgaggtggccagcacctcccccgaggacgagagcccaggcagcaaccccgagcccgacgccacccccttccaggagggcctgcgcaccttcgaccagctggacgccatcagcagcctgcccacccccagcgacatcttcgtgagctacagcacctttcccggcttcgtgagctggcgcgatcccaagtccggctcttggtatgtggagaccctggacgacatctttgagcagtgggctcatagcgaggacctgcagagcctgctgctgcgcgtggccaatgccgtgagcgtgaagggcatctacaagcagatgccaggctgcttcaacttcctgcggaagaagctgttcttcaagaccagcgcctcctga

SEQ ID NO: 25 - DNA, 3253 bp, Artificial Sequence
  source: /note=“Description of Artificial Sequence Synthetic polynucleotide”
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggccagctacccctgtcaccagcacgccagcgccttcgaccaggccgccagaagcaggggccacagcaaccggcggaccgccttaagacccaggcggcagcaggaagccaccgaagtccggctggaacagaagatgcccaccctgctgcgggtgtacatcgacggcccccacggcatgggcaagaccaccaccacccagctgctggtggccctgggcagccgggacgacatcgtgtacgtgcccgagcccatgacctactggcaggtgctgggcgccagcgagaccatcgccaacatctacaccacacagcacaggctggaccagggcgagatctctgccggcgacgccgccgtggtgatgaccagcgcccagatcacaatgggcatgccctacgccgtgaccgacgccgtgctggcccctcacgtgggcggcgaggccggctctagccacgcccctccccctgccctgaccctgatcttcgaccggcaccccatcgcccacctgctgtgctaccctgccgccagatacctgatgggcagcatgaccccccaggccgtgctggccttcgtggccctgatcccccccaccctgcccggcaccaacatcgtgctgggagccctgcccgaggaccggcacatcgaccggctggccaagcggcagagacccggcgagcggctggacctggccatgctggccgccatccggcgggtgtacggcctgctggccaacaccgtgagatacctgcagggcggagggtcttggtgggaggactggggccagctgtccggcaccgccgtgccacctcagggcgccgagccccagagcaatgccggccctcggccccacatcggcgacaccctgtttaccctgttcagagcccccgagctgctggcccccaacggcgacctgtacaacgtgttcgcctgggccctggacgtgctggccaagaggctgcggcccatgcacgtgttcatcctggactacgaccagagccctgccggctgcagggacgccctgctgcagctgaccagcggcatggtgcagacccacgtgaccacccccggcagcatccccaccatctgcgacctggcccggaccttcgcccgggagatgggcgaggccaacggaagcggagctactaacttcagcctgctgaagcaggctggcgacgtggaggagaaccctggacctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 26 - DNA, 2599 bp, Artificial Sequence
  source: /note=“Description of Artificial Sequence Synthetic polynucleotide”
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggaataacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggccagctacccctgtcaccagcacgccagcgccttcgaccaggccgccagaagcaggggccacagcaaccggcggaccgccttaagacccaggcggcagcaggaagccaccgaagtccggctggaacagaagatgcccaccctgctgcgggtgtacatcgacggcccccacggcatgggcaagaccaccaccacccagctgctggtggccctgggcagccgggacgacatcgtgtacgtgcccgagcccatgacctactggcaggtgctgggcgccagcgagaccatcgccaacatctacaccacacagcacaggctggaccagggcgagatctctgccggcgacgccgccgtggtgatgaccagcgcccagatcacaatgggcatgccctacgccgtgaccgacgccgtgctggcccctcacgtgggcggcgaggccggctctagccacgcccctccccctgccctgaccctgatcttcgaccggcaccccatcgcccacctgctgtgctaccctgccgccagatacctgatgggcagcatgaccccccaggccgtgctggccttcgtggccctgatcccccccaccctgcccggcaccaacatcgtgctgggagccctgcccgaggaccggcacatcgaccggctggccaagcggcagagacccggcgagcggctggacctggccatgctggccgccatccggcgggtgtacggcctgctggccaacaccgtgagatacctgcagggcggagggtcttggtgggaggactggggccagctgtccggcaccgccgtgccacctcagggcgccgagccccagagcaatgccggccctcggccccacatcggcgacaccctgtttaccctgttcagagcccccgagctgctggcccccaacggcgacctgtacaacgtgttcgcctgggccctggacgtgctggccaagaggctgcggcccatgcacgtgttcatcctggactacgaccagagccctgccggctgcagggacgccctgctgcagctgaccagcggcatggtgcagacccacgtgaccacccccggcagcatccccaccatctgcgacctggcccggaccttcgcccgggagatgggcgaggccaactaa

SEQ ID NO: 27 - DNA, 66 bp, Artificial Sequence
  source: /note=“Description of Artificial Sequence Synthetic oligonucleotide”
ggaagcggagctactaacttcagcctgctgaagcaggctggcgacgtggaggagaaccctggacct

SEQ ID NO: 28 - DNA, 1665 bp, Artificial Sequence
  source: /note=“Description of Artificial Sequence Synthetic polynucleotide”
atgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtaccgccttgcccagggtgggccccgtgaggcccactggccaggactggaatcacaccccccagaagacagaccatccatctgccctgctcagagaccccccggagccaggctctcccaggatctcatcactgcgcccccagggcctcagcaacccctccaccctctctgctcagccacagctttccagaagccactcctcgggcagcgtgctgccccttggggagctggagggcaggaggagcaccagggatcggaggagccccgcagagccagaaggaggaccagcaagtgaaggggcagccaggcccctgccccgttttaactccgttcctttgactgacacaggccatgagaggcagtccgagggatccttcagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctgactcaggatgacagacaggtggaactgccagtgtag

(19) United States
(12) Patent Application Publication
(10) Pub. No.: US 2024/0060047 A1
(43) Pub. Date: Feb. 22, 2024

(54) CELLS WITH SUSTAINED TRANSGENE EXPRESSION

(71) Applicant: BlueRock Therapeutics LP, Cambridge, MA (US)

(72) Inventors: Chew-Li SOH, Brooklyn, NY (US); Mark James TOMISHIMA, Chatham, NJ (US); Dan Charles WILKINSON, Jr., Fremont, CA (US); Conor Brian McAULIFFE, Summit, NJ (US)

(73) Assignee: BlueRock Therapeutics LP, Cambridge, MA (US)

(21) Appl. No.: 17/766,380

(22) PCT Filed: Oct. 9, 2020
(86) PCT No.: PCT/US2020/055158

§ 371 Date: Apr. 4, 2022

Related U.S. Application Data
(60) Provisional application No. 62/913,062, filed on Oct. 9, 2019.

(51) Int. Cl. C12N 5/077; A61K 35/34; C12N 9/02; C12N 9/12

(52) U.S. Cl. CPC C12N 5/0657; A61K 35/34; C12N 9/0008; C12N 9/1211; C12Y 102/01012; C12Y 207/01021; C12N 2506/45; C12N 2510/00

(57) ABSTRACT

Provided herein are genetically engineered mammalian (e.g., human) cells that express one or more transgenes at a sustained expression level. Also provided are methods of making and using the cells.

### CROSS REFERENCE TO RELATED APPLICATION

The present application claims priority from U.S. Provisional Application No. 62/913,062, filed on Oct. 9, 2019, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Oct. 9, 2020, is named 025450_WO009_SL.txt and is 29,071 bytes in size.

### BACKGROUND OF THE INVENTION

Cell therapy provides great promise for the treatment of a variety of diseases and conditions. In cell therapy, autologous or allogeneic cells are transplanted into a patient to replace or repair defective or damaged tissue or cells. Many different types of cells may be used, such as pluripotent stem cells (PSCs), multipotent stem cells (e.g., hematopoietic stem cells and mesenchymal stem cells), or differentiated cells (e.g., dopaminergic neurons, lymphocytes, cardiomyocytes, and pancreatic islet cells). Potential applications of cell therapy include treatment of cancers, autoimmune diseases, and regeneration of damaged tissues in, for example, joints, the heart, and the central and/or peripheral nervous system.

Therapeutic cells in cell therapy may be genetically modified, with a transgene stably integrated into their genome. The transgene, when expressed, may introduce to the modified cells a novel feature such as a protein not normally present. However, stable long-term transgene expression within a cell or organism remains a challenge in the field. A transgene may be subject to pre-existing or developmentally regulated gene expression patterns of a target cell. Such patterns can override the signals from transgenic regulatory elements through, for example, DNA methylation and histone modifications of the genome, resulting in chromatin remodeling and transgene silencing.

Similar problems exist for integration of transgenes into loci of certain ubiquitously expressed genes such as housekeeping genes. A number of genes are ubiquitously expressed in all human tissues. Due to this uniformity of expression, the promoters from these genes may seem to be prime candidates for gene engineering if sustained transgene expression is desired (Kao et al., Stem Cell Rep. (2016) 9(3):518-26). However, some of these genes have been found not as uniformly expressed in all known cell phenotypes as previously thought (de Jonge et al., PLoS One (2007) 2(9):e898). Therefore, using the promoters of these housekeeping genes for transgene expression may ultimately lead to low or negligible levels of transgene expression.

Thus, there remains a need to identify transgene integration sites that permit sustained transgene expression in PSCs and PSC-derived cells.

### SUMMARY OF THE INVENTION

The present disclosure provides a genetically modified mammalian cell comprising a transgene at a sustained transgene expression locus (STEL) in the genome, wherein the transgene is expressed at a detectable level. In some embodiments, the expression level of the transgene does not change more than 40%, more than 30%, more than 20%, or more than 10% (i) over five or more, ten or more, or 15 or more passages, or (ii) as the cell state changes, wherein the cell state is optionally state of pluripotency and/or differentiation.

The STEL site may be, e.g., one of the gene loci listed in Table 1 below. In some embodiments, the STEL is a gene locus having a mean normalized expression of more than 3.30, more than 3.50, more than 3.75, more than 4.00, more than 4.10, more than 4.20, more than 4.30, more than 4.50, more than 4.60, more than 4.70 as set forth in the table.

In some embodiments, the STEL is a gene locus that encodes a protein involved in one or more of: ribonucleoprotein complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and protein binding.

In some embodiments, the STEL is a gene encoding a ribosomal protein, such as an RPL gene (e.g., RPL13A, RPLP0, RPL10, RPL13, RPS18, RPL3, RPLP1, RPL15, RPL41, RPL11, RPL32, RPL18A, RPL19, RPL28, RPL29, RPL9, RPL8, RPL6, RPL18, RPL7, RPL7A, RPL21, RPL37A, RPL12, RPL5, RPL34, RPL35A, RPL30, RPL24, RPL39, RPL37, RPL14, RPL27A, RPLP2, RPL23A, RPL26, RPL36, RPL35, RPL23, RPL4, and RPL22) or an RPS gene (e.g., RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS16, RPS25, RPS15, RPS20, and RPS11); a gene encoding a mitochondria protein (e.g., MT-CO1, MT-CO2, MT-ND4, MT-ND1, and MT-ND2), a gene encoding an actin protein (e.g., ACTG1 and ACTB); a gene encoding a eukaryotic translation factor (e.g., EEF1A1, EEF2, and EIF1); a gene encoding a histone (e.g., H3F3A and H3F3B); or a gene selected from FTL, FTH1, TPT1, TMSB10, GAPDH, PTMA, GNB2L1, NACA, YBX1, NPM1, FAU, UBA52, HSP90AB1, MYL6, SERF2, and SRP14. In particular embodiments, the STEL is a GAPDH, RPL13A, RPL 7, or RPLP0 gene locus.

In some embodiments, the transgene is inserted into the 3′ untranslated region of the gene locus. In some embodiments, the transgene sequence is linked in frame to the STEL gene sequence through a coding sequence for a self-cleaving peptide. In some embodiments, the transgene sequence is linked to the STEL gene sequence through an internal ribosomal entry site (IRES).

In some embodiments, the transgene encodes a therapeutic protein, an immunomodulatory protein, a reporter protein, or a safety switch signal (e.g., a suicide gene).

In some embodiments, the genetically modified mammalian cell is a human cell and may be, e.g., a PSC (e.g., an embryonic stem cell or an induced PSC), or a differentiated cell. In some embodiments, the differentiated cell is (i) an immune cell, optionally selected from a T cell, a T cell expressing a chimeric antigen receptor (CAR), a suppressive T cell, a myeloid cell, a dendritic cell, and an immunosuppressive macrophage; (ii) a cell in the nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron; (iii) a cell in the cardiovascular system, optionally selected from a cardiomyocyte, an endothelial cell, and a nodal cell; or (iv) a cell in the metabolic system, optionally selected from a hepatocyte, a cholangiocyte, and a pancreatic beta cell.

In another aspect, the present disclosure provides a method of treating a human patient in need thereof, comprising introducing the present genetically modified human cells. Also provided are the genetically modified human cells for use in treating a human patient in need thereof, and the use of the genetically modified human cells for the manufacture of a medicament for treating a human in need thereof

In yet another aspect, the present disclosure provides a method of generating a genetically modified mammalian cell described herein, comprising providing a cultured mammalian cell and introducing a transgene of interest into a STEL site in the genome of the cultured cell. In some embodiments, the transgene is introduced to the genome of the cell through CRISPR gene editing (e.g., CRISPR-Cas9 gene editing).

In some embodiments, the engineered cell of the present disclosure is a pluripotent stem cell (PSC), such as an embryonic stem cell (e.g., a human embryonic stem cell) or an induced PSC (e.g., a human induced PSC). In some embodiments, the engineered cell is a differentiated cell, such as an immune cell (e.g., a T cell, a T cell expressing a chimeric antigen receptor (CAR), a myeloid cell, or a dendritic cell), an immunosuppressive cell (e.g., a suppressive T cell, or an immunosuppressive macrophage), a cell in the nervous system (e.g., a dopaminergic neuron, a microglial cell, an oligodendrocyte, an astrocyte, a cortical neuron, a spinal or oculomotor neuron, an enteric neuron, a Placode-derived cell, a Schwann cell, or a trigeminal or sensory neuron), a cell in the cardiovascular system (e.g., a cardiomyocyte, an endothelial cell, or a nodal cell), a cell in the metabolic system (e.g., a hepatocyte, a cholangiocyte, or a pancreatic beta cell), or a cell in the human ocular system, optionally selected from a retinal pigment epithelial cell, a photoreceptor cone cell, a photoreceptor rod cell, a bipolar cell, and a ganglion cell.

In another aspect, the present disclosure provides a method of treating a human patient in need thereof, comprising introducing the genetically modified human cell of the present disclosure to the patient. In some embodiments, where the introduced engineered cell contains a suicide gene, the method may further comprise administering an activator of the suicide gene at a desired time.

In some embodiments, the human patient is in need of immune suppression, and the genetically modified immune cell is an immunosuppressive cell, a suppressive T cell, or an immunosuppressive macrophage. In some embodiments, the human patient is in need of graft transplantation, or has inflammation (e.g., neuroinflammation), an autoimmune disease, or cancer. In some embodiments, the human patient is in need of cell therapy for, e.g., damaged or degenerated tissue (e.g., the brain tissue, the heart tissue, the muscle tissue, the joint, or tissue involved in metabolism).

In yet another aspect, the present disclosure provides a method of generating the genetically modified recombinant human cell described herein, comprising providing a cultured human cell and introducing the exogenous sequence and/or suicide gene into the genome of the cultured human cell. In some embodiments, the introducing step is performed through homologous recombination with or without nuclease-mediated gene editing (e.g., ZFN, TALEN or CRISPR-Cas9 or CRISPR-cpfl). Non-homologous end joining can also be used to target the transgene.

Also provided herein are genetically modified human cells, as described herein, for use in treating a human patient in need thereof in one of the present treatment methods. Also provided is the use of genetically modified human cells, as described herein, for the manufacture of a medicament for treating a human in need thereof in one of the present treatment methods. Also provided are articles of manufacture, such as kits, containing the genetically modified human cells described herein.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a panel of UMAP plots showing the ubiquity of expression of four different putative STEL genes in the context of the cell types included in the analysis. Cell types: dopaminergic neurons, microglia, pluripotent stem cells, and ventricular cardiomyocytes. Panel a: UMAP plot showing the identity and clustering of the four cell types included in the analysis. Panel b: UMAP plots showing the expression profile of GAPDH, RPL7, RPLP0, and RPL13A.

FIG. 2 is a diagram illustrating the integration of an enhanced green fluorescent protein (EGFP) transgene into the human GAPDH, RPL13A, RPLP0, or RPL7 gene locus. The coding sequence of the targeted endogenous gene was linked to the EGFP coding sequence through the coding sequence for a self-cleaving PQR peptide.

FIG. 3 is a cytometric plot showing EGFP expression levels in PSCs homozygous or heterozygous for the EGFP transgene targeted to the GAPDH or RPL13A gene locus. Unedited PSCs (PSCs not containing the transgene) were used as a negative control.

FIG. 4 is a cytometric plot showing EGFP expression levels in PSCs heterozygous for the EGFP transgene targeted to the RPLP0 gene locus. Unedited PSCs (PSCs not containing the transgene) were used as a negative control.

FIG. 5 is a qPCR histogram showing that EGFP expression was detected in GAPDH-targeted EGFP edited heterozygous and homozygous PSCs but not in unedited PSCs (PSCs not containing the transgene) on a weekly basis for up to eight weeks.

FIG. 6 is a qPCR histogram showing that EGFP expression was detected in RPL/3A-targeted EGFP edited heterozygous and homozygous PSCs but not in unedited PSCs (PSCs not containing the transgene) on a weekly basis for up to eight weeks.

FIG. 7 is a cytometric plot showing EGFP expression levels in PSC-derived cells homozygous or heterozygous for the EGFP transgene targeted to the GAPDH or RPL13A gene locus. After gene editing, the cells were assayed after 16 days of differentiation into dopaminergic neurons.

FIG. 8 is a pair of cytometric plots showing EGFP expression levels in PSCs or PSC-derived cells heterozygous for the EGFP transgene targeted to the GAPDH or RPL13A gene locus. After gene editing, the cells were assayed after 12 days of differentiation into cardiomyocytes. Unedited PSCs (PSCs not containing the transgene) were used as a negative control.

FIG. 9 is a diagram illustrating the integration of an HLA-G6 transgene into the human GAPDH or RPL13A gene locus. The coding sequence of the targeted endogenous gene was linked to the HLA-G6 coding sequence through the coding sequence for a self-cleaving PQR peptide.

FIG. 10 is a Western blot photograph showing that HLA-G6 was detected by an HLA-G5/G6-specific antibody in cell culture supernatants of GAPDH-targeted HLA-G6 edited PSCs and JEG-3 cells (positive control). Unedited (“wildtype”) PSCs were used as a negative control.

FIG. 11 is a fluorescence resonance energy transfer (FRET) assay histogram showing that HLA-G6 was detected in cell culture supernatants of GAPDH-targeted HLA-G6 edited PSCs and JEG-3 cells (positive control). Unedited (“wildtype”) PSCs were used as a negative control.

FIG. 12 is a FRET assay histogram showing that HLA-G6 was detected in cell culture supernatant of RPL/3A-targeted HLA-G6 edited PSCs but not in unedited (“wildtype”) PSCs.

FIG. 13 is a panel of cytometric plots showing HLA-G expression in PSCs edited for the HLA-G6 transgene targeted to the GAPDH or RPL13A gene locus and B2M knockout (KO). HLA-G expression can be detected after 1 week and 8 weeks of analysis in edited PSCs but not in unedited PSCs (PSCs not containing the transgene).

FIG. 14 is a diagram illustrating the integration of an anti-tau scFv transgene into the human GAPDH gene locus. The coding sequence of the targeted endogenous gene was linked to the scFv coding sequence through the coding sequence for a self-cleaving PQR peptide. SP: signal peptide coding sequence. PL: peptide linker coding sequence. HA: hemagglutinin A tag coding sequence.

FIG. 15 is a Western blot photograph showing that the anti-tau scFv was detected in neat and concentrated cell culture supernatants and cell lysates of GAPDH-targeted scFv edited PSCs. Unedited (“wildtype”) PSCs were used as a negative control.

FIG. 16 is a diagram illustrating the integration of two components of the RapaCasp9 transgene into the human GAPDH gene locus. The coding sequence of the targeted endogenous gene is linked to each RapaCasp9 coding sequence through the coding sequence of a self-cleaving PQR peptide. L1: FRB peptide linker coding sequence. L2: FKBP12 peptide linker coding sequence. truncCasp9: truncated Caspase 9 with the CARD domain removed.

FIG. 17 is a panel of cytometric dot plots showing detection of cleaved Caspase 3 following addition of 5 nM or 10 nM rapamycin to PSCs biallelically edited for the RapaCasp9 transgene targeted to the GAPDH gene locus. Cells were analyzed after rapamycin treatment for 1, 2, 4, or 24 hours and compared to untreated edited PSCs that served as a negative control.

FIG. 18 is a panel of two cytometric dot plots showing detection of PD-L1 and CD47 co-staining in PSCs biallelically edited for a PD-L1-based transgene and a CD47-based transgene targeted to the human GAPDH gene locus.

FIG. 19 is an ELISA immunoassay histogram showing that CSF1 was detected in the cell culture supernatant of three different GAPDH-targeted CSF1 edited human PSC lines but not in unedited PSCs.

FIG. 20A is a diagram showing a transgene integration site at the AAVS1 locus. The transgene encodes PD-L1 and HSV-TK. The coding sequences for the two proteins are separated in frame by a P2A coding sequence. The transgene is under the control of an EF1α promoter.

FIG. 20B is a panel of two cytometric plots showing PD-L1 expression levels from the transgene shown in FIG. 20A in undifferentiated edited human PSCs and cardiomyocytes differentiated from the PSCs.

### DETAILED DESCRIPTION OF THE INVENTION

The present invention is based on the discovery that certain loci in the genome, termed “sustained transgene expression loci” (STEL) herein, are more resistant to silencing than non-STEL loci. Resistance to silencing may be observed, for example, as the STEL-engineered cells are cultured over time (e.g., over days in culture, optionally including one or more cell passages) or as the cell fate changes (e.g., differentiation from pluripotent stem cells to lineage-specific cells). When a transgene is inserted into such a locus, expression of the transgene can be sustained, making transgene-dependent cell therapy much more efficacious.

Accordingly, the present disclosure provides methods of obtaining genetically modified mammalian cells (e.g., human) in which an exogenously introduced transgene is expressed at a stable, sustained level over a period of time or as the cells differentiate. These methods are especially advantageous when applied to PSCs engineered for use in cell therapy. Genetically modified PSCs obtained by the present methods do not lose transgene expression over time in culture and/or as the cells are differentiated into one or more cells.

In some embodiments, the expression level of the transgene in the modified cells does not change by more than 50%, more than 40%, more than 35%, more than 30%, more than 25%, more than 20%, more than 15%, more than 10%, or more than 5% over one or more cell culture passages, as compared to the expression level of the transgene prior to the one or more passages. The one or more passages may be, e.g., two or more, three or more, four or more, five or more, six or more, seven or more, eight or more, nine or more, ten or more, or 15 or more passages.

In some embodiments, the expression level of the transgene in the modified cells does not change by more than 50%, more than 40%, more than 35%, more than 30%, more than 25%, more than 20%, more than 15%, more than 10%, or more than 5% as the cell state changes in the cells, as compared to the expression level of the transgene prior to the cell state change. A cell state may be, e.g., a cell's pluripotency, biological activity, phenotype, or differentiation status.

The expression level of a gene (e.g., a transgene or an endogenous gene) can be determined by any method suitable for the particular gene. For example, the level of RNA (e.g., by RT-PCR) or protein (e.g., by FRET, ELISA, cytometric analysis, and Western blot) expressed from the gene can be measured.

To date, transgenes are most commonly targeted to safe harbor sites in the genome such as the AAVS 1 locus. High level transgene expression from safe harbor loci typically requires inclusion of external promoter sequences. But different promoters vary in their ability to maintain transgene expression in specific cell populations. Increasing evidence suggests that transgene expression at AAVS 1 and other safe harbor sites is not supported in some cell lineages (e.g., dopaminergic neurons, microglia, macrophages, or T cells) and may be subject to promoter silencing. It has been observed that genetically modified human pluripotent stem cells lose transgene expression upon lineage-directed differentiation (see, e.g., Klatt et al., Hum Gene Ther. (2020) 31(3 -4): 199-210; Ordovas et al., Stem Cell Rep. (2015) 5:918-31). The present disclosure provides methods of transgene expression that circumvent this problem and will greatly facilitate development of cell therapy.

I. Sustained Transgene Expression Loci

The sustained transgene expression loci (STEL) of the present disclosure include, without limitation, certain housekeeping genes that are active in multiple cell types such as those involved in gene expression (e.g., transcription factors and histones), cellular metabolism (e.g., GAPDH and NADH dehydrogenase), or cellular structures (e.g., actin), or those that encode ribosomal proteins (e.g., large or small ribosomal subunits, such as RPL13A, RPLP0 and RPL7). Additional examples of STEL are shown in Table 1 below. These proteins include those that form ribonucleoprotein complex, focal adhesion, cell-substrate adherens junction, cell-substrate junction, cell anchoring, extracellular exosome, extracellular vesicle, intracellular organelle, or anchoring junction. Some of the proteins are involved in RNA binding, nucleic acid binding (e.g., rRNA or mRNA binding), or protein binding.

In some embodiments, a STEL site is the locus of an endogenous gene that is robustly and consistently expressed in the pluripotent state as well as during differentiation (e.g., as examined by single-cell RNA sequencing (scRNAseq) analysis). For example, the expression level of the endogenous gene does not change (e.g., decrease) by more than 50%, more than 40%, more than 35%, more than 30%, more than 25%, more than 20%, more than 15%, more than 10%, or more than 5% over five or more, ten or more, or 15 or more passages or as the cell state changes (e.g., state of pluripotency and/or differentiation).

In some embodiments, the STEL is a ribosomal protein gene locus, such as an RPL or RPS gene locus. Examples of RPL genes are RPL10, RPL13, RPS18, RPL3, RPLP 1, RPL13A, RPL15, RPL41, RPL11, RPL32, RPL18A, RPL19, RPL28, RPL29, RPL9, RPL8, RPL6, RPL18, RPL7, RPL7A, RPL21, RPL37A, RPL12, RPL5, RPL34, RPL35A, RPL30, RPL24, RPL39, RPL37, RPL14, RPL27A, RPLP2, RPLP0, RPL23A, RPL26, RPL36, RPL35, RPL23, RPL4, and RPL22 . Examples of RPS genes are RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS16, RPS25, RPS15, RPS20, and RPS11.

In some embodiments, the STEL is a gene locus encoding a mitochondria protein. Examples of such gene loci are MT-CO1, MT-CO2, MT-ND4, MT-ND1, and MT-ND2.

In some embodiments, the STEL is a gene locus encoding an actin protein, such as ACTG1 and ACTB.

In some embodiments, the STEL is a gene locus encoding a eukaryotic translation elongation factor, such as EEF1A1 and EEF2, or a eukaryotic translation initiation factor such as EIF1.

In some embodiments, the STEL is a gene locus encoding a histone, such as H3F3A and H3F3B.

In other embodiments, the STEL is a gene locus selected from FTL, FTH1, TPT1, TMSB10, GAPDH, PTMA, GNB2L1, NACA, YBX1, NPM1, FAU, UBA52, HSP90AB1, MYL6, SERF2, and SRP14.

To introduce a transgene construct into a host cell, one can use a chemical method (e.g., calcium phosphate transfection or lipofection), a non-chemical method (e.g., electroporation or nucleofection), a particle-based method (e.g., magetofection), or viral delivery (e.g., by using viral vectors such as lentiviral vectors, adeno-associated viral (AAV) vectors, retroviral vectors, and hybrid viral vectors). The transgene may be integrated into the STEL site in a site-specific manner through, for example, a single- or double-stranded DNA break caused by ZFN, TALEN, CRISPR-cas9, CRISPR/cpfl, or another nuclease. For example, one can use various types of homologous recombination gene editing systems, where edited alleles are generated by homologous recombination between the host genome and double-stranded DNA donor molecules. Homologous recombination may be facilitated by the induction of double-stranded DNA breaks at targeted, homologous loci in the host genome and results in the exchange of the exogenous DNA donor sequence with the endogenous host genomic sequence. See, e.g., Hoshijima et al., Methods Cell Biol. (2016) 135:121-47. However, double-stranded DNA breaks are not required for homologous recombination.

Other well-known gene editing systems may also be used, such as those utilizing genome-targeting elements including a DNA-binding domain (e.g., zinc finger DNA-binding protein or a TALE DNA-binding domain), guide RNA elements (e.g., CRISPR guide RNA), and guide DNA elements (e.g., NgAgo guide DNA). Programmable gene-targeting and nuclease elements enable precise genome editing by introducing DNA breaks, such as double-stranded breaks at specific genomic loci. In some embodiments, the genome editing system is a meganuclease based system, a zinc finger nuclease (ZFN) based system, a

Transcription Activator-Like Effector-based Nuclease (TALEN) based system, a CRISPR-based system, or NgAgo-based system. In some embodiments, exogenously introduced DNA can be used to harness cellular repair mechanisms to introduce a transgene into the genome via homologous recombination.

In particular embodiments, the genome editing system is a CRISPR-based system. The CRISPR-based system comprises one or more guide RNA elements and one or more RNA-guided nucleases.

In further embodiments, the CRISPR-based system is a CRISPR-Cas system. The “CRISPR-Cas system” comprises: (a) at least one guide RNA element or a nucleic acid comprising a nucleotide sequence(s) encoding the guide RNA element, the guide RNA element comprising a targeter RNA that includes a nucleotide sequence substantially complementary to a nucleotide sequence at the one or more target genomic regions, and an activator RNA that includes a nucleotide sequence that is capable of hybridizing with the guide RNA; and (b) a Cas protein element comprising a Cas protein or a nucleic acid comprising a nucleotide sequence encoding the Cas protein. The guide RNA and activator RNA can be separate or fused together into a single RNA.

In some embodiments, the CRISPR-based system includes Class 1 CRISPR and/or Class 2 CRISPR systems. Class 1 systems employ several Cas proteins together with a CRISPR RNA (crRNA) as the targeter RNA to build a functional endonuclease. Class 2 CRISPR systems employ a single Cas protein and a crRNA as the targeter RNA. Class 2 CRISPR systems, including the type II Cas9-based system, comprise a single Cas protein to mediate cleavage rather than the multi-subunit complex employed by Class 1 systems. The CRISPR-based system also includes Class 2, Type V CRISPR system employing a Cpfl protein and a crRNA as the targeter RNA.

The Cas protein is a CRISPR-associated (Cas) double-stranded DNA nuclease. In some embodiments, CRISPR-Cas system comprises a Cas9 protein. In some embodiments, the Cas9 protein is SaCas9, SpCas9, SpCas9n, Cas9-HF, Cas9-H840A, Fokl-dCas9, or D10A nickase. The term “Cas protein,” such as Cas9 protein, includes wild type Cas protein or functional derivatives thereof (such as truncated versions or variants of the wild type Cas protein with a nuclease activity).

In some embodiments, the CRISPR-based system is a CRISPR-Cpf system. The “CRISPR-Cpf system” comprises: (a) at least one guide RNA element or a nucleic acid comprising a nucleotide sequence(s) encoding the guide RNA element, the guide RNA comprising a targeter RNA having a nucleotide sequence complementary to a nucleotide sequence at a locus of the target nucleic acid; and (b) a Cpf protein (e.g., cpfl) element or a nucleic acid comprising a nucleotide sequence encoding the Cpf protein element.

II. Transgenes

The transgene encodes a payload that may be, e.g., a therapeutic protein or a gene product that confers a desired feature to the modified cell. In some embodiments, the transgene encodes a reporter protein, such as a fluorescent protein (e.g., green fluorescent protein, red fluorescent protein, cyan fluorescent protein, yellow fluorescent protein, blue fluorescent protein, DsRED, mCherry, mKate2, and tdTomato) and an enzyme (e.g., luciferase and lacZ). A reporter gene may aid the tracking of therapeutic cells once they are implanted to a patient.

In some embodiments, the transgene encodes a therapeutic protein such as a protein deficient in a patient. Examples of such therapeutic proteins include, but are not limited to, those deficient in lysosomal storage disorders, such as alpha-L-iduronidase, arylsulfatase A, beta-glucocerebrosidase, acid sphingomyelinase, and alpha- and beta-galactosidase; and those deficient in hemophilia such as Factor VIII and Factor IX. Other examples of therapeutic proteins include, but are not limited to, antibodies or antibody fragments (e.g., scFv) such as those targeting pathogenic proteins (e.g., tau, alpha-synuclein, and beta-amyloid protein) and those targeting cancer cells (e.g., chimeric antigen receptors (CAR) targeting CD19, CD20, and tumor antigens).

In some embodiments, the transgene encodes a protein involved in immune regulation, or an immunomodulatory protein. Examples of such proteins are HLA-G, HLA-E, CD47, PD-L1, CTLA-4, M-CSF, IL-4, IL-6, IL-10, IL-11, IL-13, TGF-01, and various isoforms thereof. By way of example, the transgene may encode an isoform of HLA-G (e.g., HLA-G1, -G2, -G3, -G4, -G5, -G6, or -G7) or HLA-E; allogeneic cells expressing such a nonclassical MHC class I molecule may be less immunogenic and better tolerated when transplanted into a human patient who is not the source of the cells, making “universal” cell therapy possible. See also detailed description below.

In some embodiments, the transgene encodes a safety switch signal. In cell therapy, a safety switch can be used to stop proliferation of the genetically modified cells when their presence in the patient is not desired, for example, if the cells do not function properly or if the therapeutic goal has been achieved. A safety switch may, for example, be a so-called suicide gene, which upon administration of a pharmaceutical compound to the patient, will be activated or inactivated such that the cells enter apoptosis. A suicide gene may encode an enzyme not found in humans (e.g., a bacterial or viral enzyme) that converts a harmless substance into a toxic metabolite in the human cell. Examples of suicide genes include, without limitation, genes for thymidine kinases, cytosine deaminases, intracellular antibodies, telomerases, toxins, caspases (e.g., iCaspase9) and HSV-TK, and DNases. See, e.g., Zarogoulidis et al., J Genet Syndr Gene Ther. (2013) doi:10.4172/2157-7412.1000139. In some embodiments, the suicide gene may be a thymidine kinase (TK) gene from the Herpes Simplex Virus (HSV) and the suicide TK gene becomes toxic to the cell upon administration of ganciclovir, valganciclovir, famciclovir, or the like to the patient.

In some embodiments, the safety switch may be a rapamycin-inducible human Caspase 9-based (RapaCasp9) cellular suicide switch in which a truncated caspase 9 gene, which has its CARD domain removed, is linked after either the FRB (FKBP12-rapamycin binding) domain of mTOR, or FKBP12 (FK506-binding protein 12). Addition of the drug rapamycin enables heterodimerization of FRB and FKBP12 which subsequently causes homodimerization of truncated caspase 9 and induction of apoptosis.

In some embodiments, the transgene encodes a payload that is not a polypeptide. For example, the transgene may encode a miRNA that can selectively eliminate cells based on gene expression patterns. The transgene also may encode lncRNA or other RNA switches that can control cellular behavior in a desirable way.

III. Expression of Transgenes at STEL Sites

The transgene may be transcribed together with the endogenous gene at the STEL site, under the transcriptional control of the endogenous promoter, into one mRNA, and then the RNA sequence for each gene is translated separately through the use of an internal ribosome entry site (IRES) in the mRNA. In yet another approach, the transgene may be inserted in frame into the endogenous gene, e.g., at the 3′ end of the endogenous gene, but separated from the endogenous gene sequence by the coding sequence for a self-cleaving peptide, which causes ribosomal skipping during translation. This arrangement results in production of two separate polypeptides—the payload encoded by the transgene and the polypeptide encoded by the endogenous gene. Examples of self-cleaving peptides are 2A peptides, which are viral derived peptides with a typical length of 18-22 amino acids. 2A peptides include T2A, P2A, E2A, F2A, and PQR (Lo et al., Cell Reports (2015) 13:2634-2644). By way of example, P2A is a peptide of 19 amino acids; after the cleavage, a few amino acid residues from the P2A are left on the upstream gene and a proline is left at the beginning of the second gene. See also the Examples below for the use of a PQR peptide. In other embodiments, the STEL gene and the transgene are transcribed into a single mRNA and expressed as a fusion protein.

In some embodiments, the transgene construct may introduce additional regulatory sequences, such as a transcription termination sequence (e.g., polyadenylation (polyA) site such as a SV40 polyA site) and a sequence that enhances gene expression or RNA stability (e.g., a WPRE element), to the targeted locus. To further ensure sustained expression of the transgene, suitable transcription regulatory elements also may be introduced via the transgene construct into the targeted STEL site. Such elements include, without limitation, a ubiquitous chromatin opening element (UCOE) placed upstream of the promoter, and chromatin insulators that create functional boundaries. Chromatin insulators (e.g., chicken beta globin gene cluster (cHS4) and ArsI) can be enhancer blocking or barrier insulators that prevent silencing heterochromatin from spreading into the transgene.

IV. Genetically Modified Cells

The present disclosure provides mammalian (e.g., human, non-human primate, rodent, or murine) cells containing one or more transgenes at one or more STEL sites in the genome. The cells, such as human cells, may be engineered in vitro, in vivo, or ex vivo by gene editing methods such as those described herein. A variety of human cell types may be engineered to express a transgene of interest. In some embodiments, the cells to be engineered are pluripotent stem cells, such as human embryonic stem cells (hESCs) or human induced pluripotent stem cells (iPSCs), which can be subsequently induced to differentiate into a desired cell type, referred to herein as PSC-derivatives, PSC-derivative cells, or PSC-derived cells. In still other embodiments, the cells to be engineered are differentiated cells (e.g., partially or terminally differentiated cells). Partially differentiated cells may be, for example, tissue-specific progenitor or stem cells, such as hematopoietic progenitor or stem cells, skeletal muscle progenitor or stem cells, cardiac progenitor or stem cells, neuronal progenitor or stem cells, and mesenchymal stem cells.

As used herein, the term “pluripotent” or “pluripotency” refers to the capacity of a cell to self-renew and to differentiate into cells of any of the three germ layers: endoderm, mesoderm, or ectoderm. “Pluripotent stem cells” or “PSCs” include, for example, ESCs derived from the inner cell mass of a blastocyst or derived by somatic cell nuclear transfer, and iPSCs derived from non-pluripotent cells.

As used herein, the terms “embryonic stem,” “ES” cells, and “ESCs” refer to pluripotent stem cells obtained from early embryos. In some embodiments, the term excludes stem cells involving destruction of a human embryo; that is, the ESCs are obtained from a previously established ESC line.

The term “induced pluripotent stem cell” or “iPSC” refers to a type of pluripotent stem cell artificially prepared from a non-pluripotent cell, such as an adult somatic cell, partially differentiated cell or terminally differentiated cell, such as a fibroblast, a cell of hematopoietic lineage, a myocyte, a neuron, an epidermal cell, or the like, by introducing or contacting the cell with one or more reprogramming factors. Methods of producing iPSCs are known in the art, and include, for example, inducing expression of one or more genes (e.g., POU5F1/OCT4 (Gene ID: 5460) in combination with, but not restricted to, SOX2 (Gene ID: 6657), KLF4 (Gene ID: 9314), c-MYC (Gene ID: 4609, NANOG (Gene ID: 79923), and/or LIN28/LIN28A (Gene ID: 79727)). Reprogramming factors may be delivered by various means (e.g., viral, non-viral, RNA, DNA, or protein delivery); alternatively, endogenous genes may be activated by using, e.g., CRISPR tools to reprogram non-pluripotent cells into PSCs.

Methods for inducing differentiation of PSCs into cells of various lineages are well known in the art. For example, methods for inducing differentiation of PSCs into dendritic cells are described in Slukvin et al., J Imm. (2006) 176:2924-32; and Su et al., Clin Cancer Res. (2008) 14(19):6207-17; and Tseng et al., Regen Med. (2009) 4(4):513-26. Methods for inducing PSCs into hematopoietic progenitor cells, cells of myeloid lineage, and T lymphocytes are described in, e.g., Kennedy et al., Cell Rep. (2012) 2:1722-35.

In addition to integration of a transgene of interest into a STEL site, the genetically modified human cells herein (e.g., iPSCs or ESCs) may be further engineered to improve their therapeutic potential, including making them less immunogenic in allogeneic cell therapy by knocking out one or more of their MHC class I genes (e.g., the B2M gene). The human cells may optionally include a safety switch signal (e.g., a suicide gene) in a a STEL site.

Methods of isolating and maintaining PSCs, including ESCs and iPSCs, are well known in the art. See, e.g., Thomson et al., Science (1998) 282(5391):1145-7; Hovatta et al., Human Reprod. (2003) 18(7):1404-09; Ludwig et al., Nature Methods (2006) 3:637-46; Kennedy et al., Blood (2007) 109:2679-87; Chen et al., Nature Methods (2011) 8:424-9; and Wang et al., Stem Cell Res. (2013) 11(3):1103-16.

In some embodiments, the PSCs or any of the mature or intermediate cell types derived from them may be further engineered (prior to, concurrent with, or subsequent to the STEL site engineering) for, e.g., added functions such as payload delivery and safety control.

In some embodiments, the PSCs can be differentiated into a cell type of interest for cell therapy. In some embodiments, the cells being engineered are already differentiated cell types of interest. Non-limiting examples of differentiated cell types are described below.

A. Immune Cells

The genetically modified human cells may be immune cells, including PSC-derived immune cells, such as lymphoid and lymphoid precursor cells (e.g., T cells and T cell precursor cells (irrespective of any specific T cell subtype, e.g., including regulatory T cells and T effector cells), B cells, and NK cells), myeloid and myeloid precursor cells (e.g., granulocytes, monocytes/macrophages, and microglial cells), and dendritic and dendritic precursor cells (e.g., myeloid dendritic cells and plasmacytoid dendritic cells). In some embodiments, the genetically modified cells are T cells expressing a chimeric antigen receptor (CAR) or CAR T cells. The genetically modified immune cells may also express an immunoregulatory transgene such as those described herein.

The engineered immune cells, such as immunosuppressive immune cells (e.g., regulatory T cells and immunosuppressive macrophages), can be transplanted into a patient having an autoimmune disease, including, without limitation, rheumatoid arthritis, multiple sclerosis, chronic lymphocytic thyroiditis, insulin-dependent diabetes mellitus, myasthenia gravis, chronic ulcerative colitis, ulcerative colitis, Crohn's disease, inflammatory bowel disease, Goodpasture's syndrome, systemic lupus erythematosus, systemic vasculitis, scleroderma, autoimmune hemolytic anemia, and autoimmune thyroid disease. The immune cell-based therapies may also be used in treating graft rejection in transplantation, including treatment of symptoms related to transplantation, such as fibrosis.

B. Neural Cells

The genetically modified human cells may be neural cells, including PSC-derived neural cells, including, without limitation, neurons and neuron precursor cells (irrespective of any specific neuronal subtype, e.g., including dopaminergic neurons, cortical neurons, spinal or oculomotor neurons, enteric neurons, interneurons, and trigeminal or sensory neurons) microglia and microglia precursor cells, glial cells and glial precursor cells (irrespective of any specific glial subtype, e.g., including oligodendrocytes, astrocytes, dedicated oligodendrocyte precursor cells and bipotent glial precursors, which may give rise to astrocytes and oligodendrocytes) Placode-derived cells, Schwann cells.

The engineered neural cells can be transplanted into, including, without limitation, a patient having a neurodegenerative disease. Examples of neurodegenerative diseases are Parkinson's Disease, Alzheimer's Disease, dementia, epilepsy, Lewy Body syndrome, Huntington's Disease, Spinal Muscular Atrophy, Friedreich's Ataxia, Amyotrophic Lateral Sclerosis, Batten Disease, Multiple System Atrophy, among others.

For many of these diseases, PSCs may be first directed to adopt a primitive neural cells fate through dual SMAD inhibition (Chambers et al., Nat Biotechnol. (2009) 27(3):275-80). Primitive neural cells adopt anterior characteristics, so the absence of additional signals will provide anterior/forebrain cortical cells. Caudalizing signals can be blocked to prevent paracrine signals that might otherwise generate cultures with more posterior character (for example, XAV939 can block WNT and SU5402 can block FGF signals). Dorsal cortical neurons can be made by blocking SHH activation, while ventral cortical neurons can be made through SHH activation. More caudal cell types, such as serotonergic neurons or spinal motor neurons can be made by caudalizing cultures through the addition of FGF and/or WNT signals. For some cell types, retinoic acid (another caudalizing agent) may be added to posteriorize cultures. The production of glial cell types may generally follow the same patterning of primitive neural cells before extended culture in FGF2 and/or EGF containing medium. PNS cell types may follow the same general principles but with a timely WNT signal early in the differentiation process.

The genetically modified neural cells may be introduced into the patient through a cannula placed into the damaged tissue in question. A cell preparation may be placed into supportive medium and loaded into a syringe or pipette-like device that can accurately deliver the preparation. The cannula may then be placed into a patient's nervous system, usually using stereotactic methods to precisely target delivery. Cells can then be expelled into the tissue at a rate that is compatible.

C. Cardiovascular Cells

The genetically modified human cells may be cells in the cardiovascular system, including PSC-derived cardiovascular cells, such as cardiomyocytes, cardiac fibroblasts, cardiac smooth muscle cells, epicardium cells, cardiac endothelial cells, Purkinje fibers, and pacemaker cells.

In some embodiments, cardiomyocytes prepared, enriched, or isolated by a method of the disclosure are derived from PSCs such as iPSCs. Numerous methods exist for differentiating PSCs into cardiomyocytes, for example as shown in Kattman et al., Cell Stem Cell (2011) 8(2):228-40, and as shown in WO2016131137, WO2018098597, and U.S. Pat. No. 9,453,201. Any suitable method in the art can be used with the methods herein to obtain PSC-derived cardiomyocytes modified to express a transgene at a STEL.

In some embodiments, the PSCs are incubated in one or more cardiac differentiation media. For example, the media may contain varying concentrations of bone-morphogenetic protein (BMP; such as BMP4) and activin (such as activin A). Titration of differentiation factor concentration may be performed to determine the optimal concentration necessary for achieving desired cardiomyocyte differentiation.

In some embodiments, the differentiated cardiomyocytes express one or more of cardiac troponin T (cTnT), and/or myosin light chain 2v (MLC2v). In some embodiments, the immature cardiomyocytes express one or more of troponin T, cardiac troponin I, alpha actinin and/or beta-myosin heavy chain.

D. Cells in the Metabolic System

The genetically modified human cells may be involved with the human metabolic system. For example, the cells may be cells of the gastrointestinal system (e.g., hepatocytes, cholangiocytes, and pancreatic beta cells), cells of the hematopoietic system, and cells of the central nervous system (e.g., pituitary hormone-releasing cells). By way of example, to generate pituitary hormone-releasing cells, PSCs are cultured with BMP4 and SB431542 (which block activin signaling) before the addition of SHH/FGF8 and FGF10; cells are then subjected only to SHH/FGF8 and FGF10 for an extended period before FGF8 or BMP (or both) to induce the cells to become specific hormone-releasing cells. See, e.g., Zimmer et al., Stem Cell Reports (2016) 6:858-72.

E. Cells in the Ocular System

The genetically modified human cells may be cells in the ocular system. For example, the cells may be retinal progenitor cells, retinal pigment epithelial (RPE) progenitor cells, RPE cells, neural retinal progenitor cells, photoreceptor progenitor cells, photoreceptor cells, bipolar cells, horizontal cells, ganglion cells, amacrine cells, Mueller glia cells, cone cells, or rod cells. Methods of differentiating iPSCs into RPE cells are described in, e.g., WO 2017/044483. Methods for isolating RPE cells are described in e.g., WO 2017/044488. Methods for differentiating iPSCs into neural retinal progenitor cells are described in WO 2019/204817. Methods for identifying and isolating retinal progenitor cells and RPE cells are described in e.g., WO 2011/028524.

V. Pharmaceutical Compositions and Use

The genetically engineered cells described herein may be provided in a pharmaceutical composition containing the cells and a pharmaceutically acceptable carrier. The pharmaceutically acceptable carrier may be cell culture medium that optionally does not contain any animal-derived component. For storage and transportation, the cells may be cryopreserved at <−70° C. (e.g., on dry ice or in liquid nitrogen). Prior to use, the cells may be thawed, and diluted in a sterile cell medium that is supportive of the cell type of interest.

The cells may be administered into the patient systemically (e.g., through intravenous injection or infusion), or locally (e.g., through direct injection to a local tissue, e.g., the heart, the brain, and a site of damaged tissue). Various methods are known in the art for administering cells into a patient's tissue or organs, including, without limitation, intracoronary administration, intramyocardial administration, transendocardial administration, or intracranial administration.

A therapeutically effective number of engineered cells are administered to the patient. As used herein, the term “therapeutically effective” refers to a number of cells or amount of pharmaceutical composition that is sufficient, when administered to a human subject suffering from or susceptible to a disease, disorder, and/or condition, to treat, prevent, and/or delay the onset or progression of the symptom(s) of the disease, disorder, and/or condition. It will be appreciated by those of ordinary skill in the art that a therapeutically effective amount is typically administered via a dosing regimen comprising at least one-unit dose.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

In the following Examples, gene editing was performed as described below.

### Guide RNAs and Validation

CRISPR-Cas9 gene editing was performed to insert transgenes into the intended STEL sites in the following experiments. Three guide RNAs (gRNAs) were designed computationally to target the 3′ UTR of GAPDH proximal to the stop codon. Five gRNAs were designed computationally to target the 3′ UTR of RPL13A proximal to the stop codon. These gRNAs were designed to have a low number of off-target sites and have a high predicted activity against the target sequence.

To test gRNA cutting efficiency, gRNAs complexed with Cas9 nuclease were delivered as ribonucleoproteins (RNPs) separately into human PSCs via nucleofection. At 72 hours following nucleofection, gDNA was extracted from each pool of nucleofected cells. A region around the GAPDH or RPL13A locus intended cut site was PCR-amplified using the following primers:

GAPDH F:
(SEQ ID NO: 1)
5′-TGGACCTGACCTGCCGTCTA-3′,
and
GAPDH R:
(SEQ ID NO: 2)
5′-CCCCAGACCCTAGAATAAGACAGG-3′
(amplicon size = 619 bp)
and
RPL13A F:
(SEQ ID NO: 3)
5′-AACAGTTGCATTATGATATGCCCAG-3′,
RPL13A R:
(SEQ ID NO: 4)
5′-TGCTTTCAAGCAACTTCGGGA-3′
(amplicon size = 696 bp).

PCR products were purified and Sanger sequenced using the following primers:

GAPDH:
(SEQ ID NO: 5)
5′-AAAACCTGCCAAATATGATGACA-3′
and
RPL13A:
(SEQ ID NO: 6)
5′-AAGTACCAGGCAGTGACAGC-3′

Overall cutting efficiency of each gRNA was determined by Inference of CRISPR Edits (ICE) Analysis by comparing a Sanger sequencing chromatogram from unedited cells to the Sanger sequencing chromatogram from each gRNA condition. ICE analysis determined that GAPDH gRNA with the RNA sequence of 5′-CUUCCUCUUGUGCUCUUGCU-3′ (SEQ ID NO:7) and RPL13A gRNA with the RNA sequence of 5′-GGAAGGGCAGGCAACGCAUG-3′ (SEQ ID NO:8) had the highest relative cutting efficiencies of all gRNAs tested for the respective locus.

### Knockin Generation

The chemically modified gRNAs for each selected STEL site were resuspended in nuclease-free TE buffer provided by the manufacturer and nucleofected as an RNP in complex with S. pyogenes Cas9 nuclease 2NLS (Synthego) and GAPDH- or RPL13A-targeting donor plasmid into human iPSCs. The Lonza 4D Nucleofector™ X-Unit was used for the transfections (P3 Nucleofector Solution and Nucleofector program CA-137). Individual colonies were then transferred to 96-well plates coated with recombinant truncated vitronectin via the pick-to-keep method under sterile conditions and were expanded for genetic screening and for freezing (in Essential 8 complete media+10% DMSO). Care was taken to limit the number of passages during characterization and screening to provide the lowest passage number possible.

Clones were screened for relevant knockin by 5′ and 3′ junction PCR using one primer set per pair external to the targeting construct and one primer per pair internal to the targeting construct. Clones positive for both the 5′ and 3′ junction PCR products were expanded and cryopreserved. The gDNA from each 5′ and 3′ positive clone was used as a template to generate PCR products fully spanning the integrated construct (including homology arms). These PCR products were then used to Sanger sequence the length of the integrated construct in its genomic context.

### Cell Culture Platform

iPSCs were maintained using Essential 8 medium (Thermo Fisher Scientific; Catalog# A1517001) and recombinant human vitronectin (VTN-N) (an N-terminal truncated vitronectin polypeptide). During single cell passaging and cloning procedures, Y-27632 ROCK Inhibitor was used. iPSCs were fed daily and double fed once per week. Cell cultures were maintained at 37° C. and 5% CO₂. During culture there were no significant changes in observed morphology between the knockout clones and the parental wildtype cells.

### Clonality

Immediately following electroporation for the desired genetic modification, the iPSCs were plated at a low density to ensure that single cells attached and grew independently. Each cell was allowed to grow into a colony. Once colonies reached an optimal size, each individual colony was picked and placed into a separate well. Each clone was sequence-analyzed for the gene editing event and also underwent G-banded karyotyping.

### Clone HLA-G Protein Characterization

Flow cytometry was performed using a pan-HLA-G antibody from BD Biosciences (clone 4H84) to confirm cell surface expression of HLA-G. Secretion of HLA-G6 and HLA-G5 into cell culture media was evaluated by Western blot using an HLA-G5/G6 specific antibody from Thermo Fisher Scientific (clone 5A6G7). Specifically, 4 mL of media was concentrated down to 100 μl and then tested by Western for the presence of HLA-G6 and -G5.

### Example 1: Identification of STEL Sites

In this study, we appraised single cell RNA sequencing (scRNA-seq) data collected from human PSCs and their differentiation derivatives to perform a site survey for STEL candidates. We hypothesized that putative STEL sites might be discovered using scRNA-seq data of multiple cell types. This approach would allow direct inspection of hundreds of thousands of available individual transcriptomes. In the current study, single cell RNA sequence data were collected from PSCs and three PSC-derived cell types, microglia, dopaminergic neurons, and ventricular cardiomyocytes. The data were collected from 267,058 cells with a transcriptomic depth of 28,387 unique genes. The first and foremost characteristic of a STEL site is ubiquity of expression. Genes were ranked by ubiquity of expression by first binarizing the transcript count data and then summing across cells. The sum for each gene was then divided by the total number of cells, resulting in a fraction reflecting the prevalence of that gene in the total data.

A total of 98 genes have a fractional representation of over 99% and were subsequently selected for further analysis. The selected genes were then sorted by the standard deviation of the un-binarized expression data. Genes with a standard deviation greater than 1 were removed. The remaining 94 genes were then sorted by mean expression and they were primarily ribosomal genes but also included some known housekeeping genes such as GAPDH and ACTB (Table 1).

TABLE 1
STEL Sites Identified by Single Cell RNA Sequencing

| | | | Mean Normalized |
|---|---|---|---|
| Gene | Prevalence | Standard Deviation | Expression |
| MT-CO1 | 0.99818766 | 0.79807 | 4.759424 |
| EEF1A1 | 0.9997828 | 0.480906 | 4.660437 |
| RPS2 | 0.99943835 | 0.651345 | 4.484057 |
| MT-CO2 | 0.9973714 | 0.888182 | 4.410487 |
| RPL10 | 0.999165 | 0.508622 | 4.358891 |
| RPL13 | 0.9995432 | 0.467537 | 4.279652 |
| RPS18 | 0.9991762 | 0.649357 | 4.252055 |
| RPL3 | 0.99885046 | 0.534212 | 4.237176 |
| RPLP1 | 0.9991762 | 0.625607 | 4.167585 |
| RPS19 | 0.99904513 | 0.5722 | 4.135667 |
| RPL13A | 0.9975773 | 0.815802 | 4.118467 |
| RPL15 | 0.99849844 | 0.536246 | 4.113494 |
| RPL41 | 0.9985771 | 0.63909 | 4.111437 |
| RPS14 | 0.9980416 | 0.585511 | 4.096238 |
| RPS3A | 0.9982064 | 0.547957 | 4.070287 |
| RPS12 | 0.9974575 | 0.614945 | 4.055728 |
| RPS3 | 0.9975698 | 0.612085 | 4.054404 |
| RPS4X | 0.9973676 | 0.668917 | 4.016652 |
| MT-ND4 | 0.99586606 | 0.848835 | 3.971863 |
| FTL | 0.99895155 | 0.757879 | 3.958872 |
| RPL11 | 0.99871564 | 0.52773 | 3.941935 |
| RPL7A | 0.9983599 | 0.514438 | 3.93846 |
| RPS6 | 0.99777204 | 0.63852 | 3.933282 |
| RPS23 | 0.9983037 | 0.56313 | 3.916524 |
| RPS8 | 0.9977945 | 0.549136 | 3.847651 |
| RPL32 | 0.99764097 | 0.586428 | 3.842095 |
| RPS27A | 0.99695945 | 0.580258 | 3.8081 |
| RPL18A | 0.996761 | 0.622554 | 3.78371 |
| RPL7 | 0.9927132 | 0.787255 | 3.773557 |
| FTH1 | 0.99838614 | 0.556724 | 3.767666 |
| RPL19 | 0.9979967 | 0.516219 | 3.745211 |
| ACTG1 | 0.9936119 | 0.867996 | 3.744461 |
| TPT1 | 0.9988991 | 0.475423 | 3.738384 |
| RPL28 | 0.9985209 | 0.479278 | 3.734198 |
| TMSB10 | 0.99786186 | 0.724701 | 3.730402 |
| RPL29 | 0.99746495 | 0.540111 | 3.721258 |
| GAPDH | 0.99528193 | 0.720528 | 3.709536 |
| RPS7 | 0.9970381 | 0.553198 | 3.703257 |
| RPL9 | 0.99733764 | 0.546494 | 3.7017 |
| RPL8 | 0.9972815 | 0.585284 | 3.693908 |
| RPL6 | 0.9973676 | 0.547067 | 3.692917 |
| RPL18 | 0.99819887 | 0.467107 | 3.677816 |
| RPS24 | 0.99850595 | 0.472275 | 3.67747 |
| H3F3A | 0.99548787 | 0.710396 | 3.668312 |
| PTMA | 0.9949674 | 0.771786 | 3.65728 |
| RPL21 | 0.9931213 | 0.675189 | 3.645983 |
| RPL37A | 0.99762225 | 0.565422 | 3.64233 |
| RPL12 | 0.99701566 | 0.630326 | 3.633636 |
| MT-ND1 | 0.99440944 | 0.807593 | 3.626996 |
| RPS27 | 0.99494493 | 0.620872 | 3.625914 |
| RPS9 | 0.9972702 | 0.523163 | 3.624767 |
| RPL5 | 0.99596715 | 0.567657 | 3.624523 |
| RPS15 | 0.9968284 | 0.548098 | 3.611938 |
| RPS15A | 0.995046 | 0.628772 | 3.600804 |
| RPL34 | 0.99605703 | 0.601182 | 3.594313 |
| RPL27A | 0.99752116 | 0.625041 | 3.555514 |
| ACTB | 0.99719536 | 0.761646 | 3.548128 |
| RPL35A | 0.9943907 | 0.585154 | 3.489456 |
| GNB2L1 | 0.9971205 | 0.523149 | 3.489171 |
| RPL30 | 0.99540925 | 0.512152 | 3.457516 |
| RPL24 | 0.9960121 | 0.513769 | 3.400068 |
| RPL39 | 0.99534935 | 0.597685 | 3.392521 |
| RPL37 | 0.99723655 | 0.502637 | 3.371473 |
| RPL14 | 0.99705684 | 0.508934 | 3.369644 |
| MT-ND2 | 0.99309886 | 0.776571 | 3.366508 |
| RPLP2 | 0.9960495 | 0.602278 | 3.361929 |
| RPS28 | 0.9968434 | 0.554103 | 3.348281 |
| RPS13 | 0.9931925 | 0.58795 | 3.330874 |
| RPLP0 | 0.9970381 | 0.592283 | 3.32502 |
| RPSA | 0.9936493 | 0.632439 | 3.3209 |
| RPL26 | 0.99131274 | 0.704314 | 3.319536 |
| RPS5 | 0.9913465 | 0.648693 | 3.317801 |
| RPS16 | 0.9948288 | 0.602616 | 3.309278 |
| RPL23A | 0.9924361 | 0.632758 | 3.295548 |
| NACA | 0.99612445 | 0.473038 | 3.255165 |
| RPL36 | 0.9960196 | 0.546941 | 3.243954 |
| RPL35 | 0.99485505 | 0.637233 | 3.196165 |
| H3F3B | 0.99514335 | 0.73797 | 3.193351 |
| RPS25 | 0.9933535 | 0.594325 | 3.18793 |
| RPS20 | 0.99038035 | 0.729486 | 3.118578 |
| RPS11 | 0.99536055 | 0.482263 | 3.104627 |
| RPL23 | 0.99425966 | 0.533082 | 3.103753 |
| YBX1 | 0.9908409 | 0.619021 | 3.074147 |
| RPL4 | 0.9934134 | 0.603962 | 3.02501 |
| EIF1 | 0.99651015 | 0.471994 | 3.015684 |
| NPM1 | 0.99298656 | 0.694548 | 3.013374 |
| FAU | 0.9945218 | 0.437123 | 2.972824 |
| UBA52 | 0.9927169 | 0.494786 | 2.871736 |
| RPL22 | 0.9925035 | 0.53572 | 2.861245 |
| HSP90AB1 | 0.9909907 | 0.650413 | 2.84481 |
| MYL6 | 0.9922901 | 0.615544 | 2.839953 |
| EEF2 | 0.99469405 | 0.546435 | 2.792892 |
| SERF2 | 0.992863 | 0.504087 | 2.768433 |
| SRP14 | 0.9914962 | 0.583232 | 2.75499 |

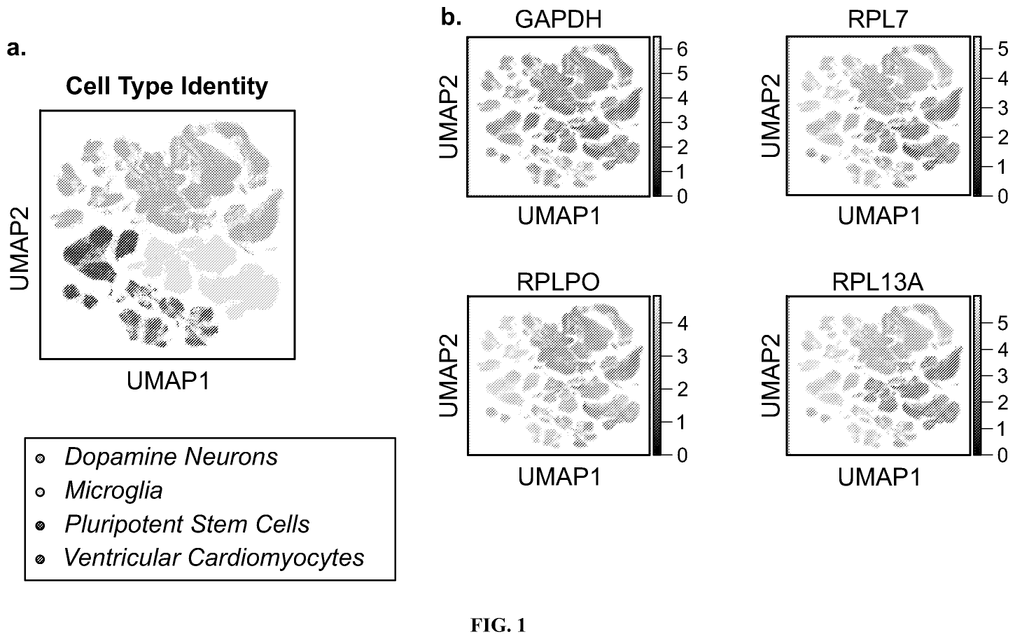

Several of these genes (GAPDH, RPLP0, RPL 7, and RPL13A) are visualized in UMAP plots shown in FIG. 1. These four gene loci were selected as STEL for the experiments described below. Other criteria we considered when finalizing selection of a STEL site from those listed above included genomic distance from oncogenes (as far as possible), published studies for proof-of-concept, and the number of pseudogenes in the loci (the fewer the better, to minimize off-target primer binding). While the above described RNA sequence method can be used to discover STEL sites, it may also be used to disqualify potential sites.

Furthermore, our scRNAseq analysis of gene expression shows that not all endogenous genes commonly used as control for gene expression analysis are STEL sites. For example, genes encoding peptidylprolyl isomerase A (PPIA; or cyclophilin A) gene, tubulin beta polypeptide (TUBB), and beta-2-microglublin (B2M) are commonly considered reliable housekeeping genes whose expression levels are used as normalizing references for RT-PCR assays of mammalian cells. But based on our data, these genes are not STEL sites because their expression levels are much more variable across cell types than the STEL genes shown in Table 1 above. Similar observations were made with other housekeeping genes commonly used as normalizing controls for RNA analysis, such as genes encoding ALAS1, GUSB, HMBS, HPRT, SDHA, TBP, and TFRC. By contrast, the ribosomal protein genes such as RPL13A and RPLP0 genes have robust expression across cell types, making them STEL sites suitable for transgene integration.

To reduce the risk of aberrant integration, a STEL is preferably not flank by an oncogene or a tumor suppressor gene. For example, the TUBB gene is in the vicinity of the MDC1 gene, a mediator of DNA repair and a known tumor suppressor gene. The TUBB gene was not chosen as a STEL site for this additional reason. The STEL sites may have splice variants, if any, and an appropriate distance from neighboring genes, that are amenable for gene editing. It may also be preferred that the STEL sites do not have a high number of pseudogenes, which may reduce transgene targeting efficiency due to sequences homologous to the targeted gene.

### Example 2: Expression of EGFP at STEL Sites in PSCs

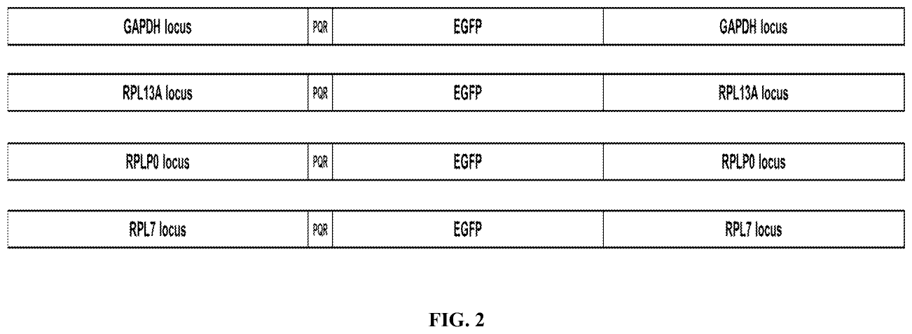

Based on the above study, we selected four STEL sites (GAPDH, RPL13A, RPLP0 and RPL 7) for testing of payload candidate expression. The expression cassette of the payload candidate was under the control of the endogenous STEL promoter. As a result, the expression of the payload candidate was linked to the expression of the endogenous STEL gene. If the STEL promoter remained active in a cell, the expression of the linked payload transgene would be expected to be sustained and constitutive. We used CRISPR-cas9 gene editing to insert a construct that expresses enhanced green fluorescent protein (EGFP) at the GAPDH, RPL13A, RPLP0 or RPL 7 gene locus (FIG. 2). The EGFP coding sequence is shown below.

(SEQ ID NO: 9)
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAGTAA

The inserted EGFP transgene was linked in frame to the endogenous STEL gene by a DNA sequence coding for a PQR sequence (Lo et al., supra) (FIG. 2). The PQR sequence is a modified 2A self-cleaving peptide that causes ribosomal skipping during translation, resulting in bicistronic expression of EGFP and the endogenous STEL gene once the PQR sequence is cleaved. The PQR nucleotide and amino acid sequences are shown below.

(SEQ ID NO: 10)
GGAAGCGGAGCGACGAATTTTAGTCTACTGAAACAAGCGGGAGACGTGGA
GGAAAACCCTGGACCT
(SEQ ID NO: 11)
GSGATNESLL KQAGDVEENP GP

Each PQR/EGFP insertion construct was also flanked by an 800 bp left homology arm and an 800 bp right homology arm carrying sequences homologous to the endogenous STEL locus. The homologous arms enabled integration of the targeting construct at the 3′ UTR of the STEL gene immediately after the last amino acid codon.

The sequences of the left and right homology arms for targeting the GAPDH locus are shown below as SEQ ID NOs:12 and 13, respectively.

(SEQ ID NO: 12)
TTGGTATCGTGGAAGGACTCATGGTATGAGAGCTGGGGAATGGGACTGAG
GCTCCCACCTTTCTCATCCAAGACTGGCTCCTCCCTGCCGGGGCTGCGTG
CAACCCTGGGGTTGGGGGTTCTGGGGACTGGCTTTCCCATAATTTCCTTT
CAAGGTGGGGAGGGAGGTAGAGGGGTGATGTGGGGAGTACGCTGCAGGGC
CTCACTCCTTTTGCAGACCACAGTCCATGCCATCACTGCCACCCAGAAGA
CTGTGGATGGCCCCTCCGGGAAACTGTGGCGTGATGGCCGCGGGGCTCTC
CAGAACATCATCCCTGCCTCTACTGGCGCTGCCAAGGCTGTGGGCAAGGT
CATCCCTGAGCTGAACGGGAAGCTCACTGGCATGGCCTTCCGTGTCCCCA
CTGCCAACGTGTCAGTGGTGGACCTGACCTGCCGTCTAGAAAAACCTGCC
AAATATGATGACATCAAGAAGGTGGTGAAGCAGGCGTCGGAGGGCCCCCT
CAAGGGCATCCTGGGCTACACTGAGCACCAGGTGGTCTCCTCTGACTTCA
ACAGCGACACCCACTCCTCCACCTTTGACGCTGGGGCTGGCATTGCCCTC
AACGACCACTTTGTCAAGCTCATTTCCTGGTATGTGGCTGGGGCCAGAGA
CTGGCTCTTAAAAAGTGCAGGGTCTGGCGCCCTCTGGTGGCTGGCTCAGA
AAAAGGGCCCTGACAACTCTTTTCATCTTCTAGGTATGACAACGAATTTG
GCTACAGCAACAGGGTGGTGGACCTCATGGCCCACATGGCCTCCAAAGAG
(SEQ ID NO: 13)
CCCTGGACCACCAGCCAAAGCAAGAGCACAAGAGGAAGAGAGAGACCCTC
ACTGCTGGGGAGTCCCTGCCACACTCAGTCCCCCACCACACTGAATCTCC
CCTCCTCACAGTTGCCATGTAGACCCCTTGAAGAGGGGAGGGGCCTAGGG
AGCCGCACCTTGTCATGTACCATCAATAAAGTACCCTGTGCTCAACCAGT
TACTTGTCCTGTCTTATTCTAGGGTCTGGGGCAGAGGGGAGGGAAGCTGG
GCTTGTGTCAAGGTGAGACATTCTTGCTGGGGAGGGACCTGGTATGTTCT
CCTCAGACTGAGGGTAGGGCCTCCAAACAGCCTTGCTTGCTTCGAGAACC
ATTTGCTTCCCGCTCAGACGTCTTGAGTGCTACAGGAAGCTGGCACCACT
ACTTCAGAGAACAAGGCCTTTTCCTCTCCTCGCTCCAGTCCTAGGCTATC
TGCTGTTGGCCAAACATGGAAGAAGCTATTCTGTGGGCAGCCCCAGGGAG
GCTGACAGGTGGAGGAAGTCAGGGCTCGCACTGGGCTCTGACGCTGACTG
GTTAGTGGAGCTCAGCCTGGAGCTGAGCTGCAGCGGGCAATTCCAGCTTG
GCCTCCGCAGCTGTGAGGTCTTGAGCACGTGCTCTATTGCTTTCTGTGCC
CTCGTGTCTTATCTGAGGACATCGTGGCCAGCCCCTAAGGTCTTCAAGCA
GGATTCATCTAGGTAAACCAAGTACCTAAAACCATGCCCAAGGCGGTAAG
GACTATATAATGTTTAAAAATCGGTAAAAATGCCCACCTCGCATAGTTTT

The sequences of the left and right homology arms for targeting the RPL13A locus are shown below as SEQ ID NOs:14 and 15, respectively.

(SEQ ID NO: 14)
TCTTAAGCCCCTCTCTTTCTCTAACAGAAAAAGCGGATGGTGGTTCCTGC
TGCCCTCAAGGTCGTGCGTCTGAAGCCTACAAGAAAGGTGAGTCCCAGCT
TACGCTGCACCATCTACTTGGGAGATTTCAGGCCTGCTGAGGGACCTGGG
GACCTGGAGCCTGGCAGATGATGTCCTTATCTCACGATGGTCTGCGGATG
TCCCTGTGGGAATGGCGACAATGCCAATGGCTTAGCTGATGCCAGGAGGC
TTGGGTGGGTGCTTTTCTAACAGGCCTGCAGAGAACAGTTGCATTATGAT
ATGCCCAGCTGTCAGTCACCTCCCAGCTCTCAACAGCTCCGGCTCTTCAG
GGTGTGGGGGCTTAGATATCCTTACAACTTCATTTGTTCACCCCCCCCCC
CCCCCCCCGCAGTTTGCCTATCTGGGGCGCCTGGCTCACGAGGTTGGCTG
GAAGTACCAGGCAGTGACAGCCACCCTGGAGGAGAAGAGGAAAGAGAAAG
CCAAGATCCACTACCGGAAGAAGAAACAGCTCATGGTGAGGCCAGGGGCT
GGTGCTGAGGGGGGCATCTCACTCCTGGACAGGCCTGGCAGGTGCCTTGC
TCACAGAGTACTCTTAACTGGCAAAGGACCAGCCGGGGTTGGGGTGGGAT
GCAGTCCATGTAATGAGGGCAATGCAACCCCTCCTGACCACCACCACCTG
CACTTATTCTTGGCAGAGGCTACGGAAACAGGCCGAGAAGAACGTGGAGA
AGAAAATTGACAAATACACAGAGGTCCTCAAGACCCACGGACTCTTAGTC
(SEQ ID NO: 15)
GCCCAATAAAGACTGTTAATTCCTCATGCGTTGCCTGCCCTTCCTCCATT
GTTGCCCTGGAATGTACGGGACCCAGGGGCAGCAGCAGTCCAGGTGCCAC
AGGCAGCCCTGGGACATAGGAAGCTGGGAGCAAGGAAAGGGTCTTAGTCA
CTGCCTCCCGAAGTTGCTTGAAAGCACTCGGAGAATTGTGCAGGTGTCAT
TTATCTATGACCAATAGGAAGAGCAACCAGTTACTATGAGTGAAAGGGAG
CCAGAAGACTGATTGGAGGGCCCTATCTTGTGAGTGGGGCATCTGTTGGA
CTTTCCACCTGGTCATATACTCTGCAGCTGTTAGAATGTGCAAGCACTTG
GGGACAGCATGAGCTTGCTGTTGTACACAGGGTATTTCTAGAAGCAGAAA
TAGACTGGGAAGATGCACAACCAAGGGGTTACAGGCATCGCCCATGCTCC
TCACCTGTATTTTGTAATCAGAAATAAATTGCTTTTAAAGAAATCTGGCG
TCTTTGCACTGTGTCTGCTGTGGAGGCAGGCCCCTGGCAAATGGGGGGTG
AGGAGCTTGAAGAGGGTAGAATGGGCTGTGCTAATATACAGAATATATGT
AACTTGCTATAAATTGAATGATCCTTTATAGACACCGTTTACAAACCAAA
GACATAAAATGTGGCCAGCAGTGCCTGGTGCTTCCTAGTTAATGTAAAGC
TGTCTCATTCTAATTCAGCTGCAAAGTATGGACCCATGCCCTGCTGCCAG
GCTGCTGTAGTCCCGGCGGTCTGTAGAGACTAGCATTTTGCAAATGATAA

The sequences of the left and right homology arms for targeting the RPLP0 locus are shown below as SEQ ID NOs:16 and 17, respectively.

(SEQ ID NO: 16)
CTGAGCTGCCAACCTGGCAATTATTGTCTGCTAAGGGTTCTCTTTATTCA
CCCTTACTTGGACTTCCTTTCCTGTAGGGAATCTCACGTAAAATGAAATC
TTCCCTCCCCCAGGGTGTCCGCAATGTTGCCAGTGTCTGTCTGCAGATTG
GCTACCCAACTGTTGCATCAGTACCCCATTCTATCATCAACGGGTACAAA
CGAGTCCTGGCCTTGTCTGTGGAGACGGATTACACCTTCCCACTTGCTGA
AAAGGTAAAAGGATCCCACCAGGACCACAGTGGGCCTGACTGTGACAAAT
TAGCAGGGTGATGTGGCCTTCTACCTTACTGCTTTTATAGTTGTATTTTA
TATAGCAGATAATTTTGTGAGGGGATATTTGAGAGGTTGGGAGGCAGGGA
AGGCGTTTCTCACTTGAGAAATGACAAGAGACCCAAAGAGGGGGTTAATG
GGCAAGAGCTGGGCCTTAGGAACCCTGCCTCACTAGGCCATACCCAAGCT
GTCCTGCTTGGGCTGCTTCTGACAGGAAAGGCTTCACACGGACTTTGATA
TTGTTGGTCCTTAAACTCTACCAAGGCAGGAGGGTGGTGGGTAATAGAGG
AGTGTGGATGACCATTTTGACCACTTCCCCCCTCCTTTCAGGTCAAGGCC
TTCTTGGCTGATCCATCTGCCTTTGTGGCTGCTGCCCCTGTGGCTGCTGC
CACCACAGCTGCTCCTGCTGCTGCTGCAGCCCCAGCTAAGGTTGAAGCCA
AGGAAGAGTCGGAGGAGTCGGACGAGGATATGGGATTTGGTCTCTTTGAC
(SEQ ID NO: 17)
TCACCAAAAAGCAACGAACTTAGCCAGTTTTATTTGCAAAACAAGGAAAT
AAATGCTTACTTCTTTAAAAAGTCTCTTGACTCTTAATTTTGTAATTTTT
TTTCCTTTTTGACACAGGGTCTGGCTGTTGCCCAGGCTGGAGTGTGGTGG
TGTAATCATAACTCACTGCACCCTTGAACTCCTGGGATCAAGGGATCCTC
GTATCTCAGCCTCCCAAGTAGCTGGGACTACAGGCACACACCATGACACT
CAGCTACTAATTTTTAAATTTTTTTTTTGTAGAGATGTTGCACAAGCTGG
TCTCAAATTCCTGGCCTCAAGGAATCCTGCCTCAGCCTCCCAAAGTGCTA
GGATTACAGGCTTGAGCCACCATGTGCCTGGCCCTTAATTTTGAGGTTTA
TAGTGCCATATGCTAGAAACGAAAGCCATGGTAAAACCAGAGCTTTGTAT
TTAGGTGTTGATGTTTGGGTATCTAAATGAAGCTACCAATCAAACATCCT
ATACAGTTTTCTAGACACAGTTGTAACTATTACACTAGAATTACTGTTTC
TATGGCTGCTGCATACTTGGAGTAGGTTTAGTGTCAGCTGAGATAGGCAC
CTGGTGGATGCTGGGGCCAGTCCCCTAGAGTAAAGTTTTTCAAACTGGGT
GGTGCTCCAACTCGGTGGTAACCAATTTATATTTTCGAGATAGTCTCAAA
TATATTTGAGACTGGGGTGCAGTGGCTTGGACTTGGCTCACTGCAACCTC
CGCCTCCTGGGTTCAAGTGATTCTCCTGCCTCAGCCTCCCAAGTAGCTGC

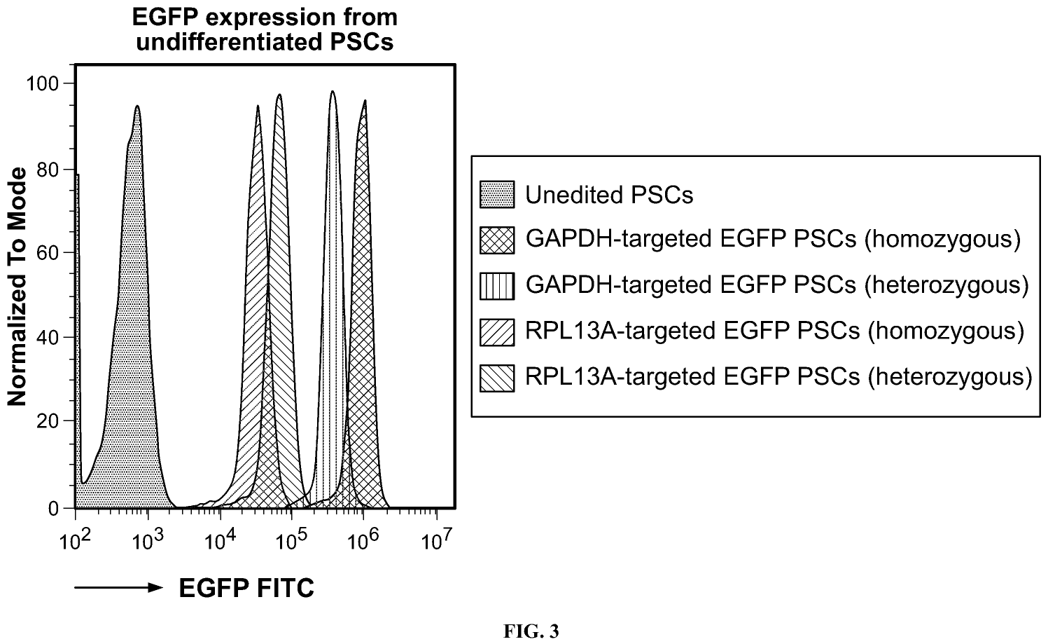

Flow cytometric analysis was performed on either undifferentiated unedited PSCs, undifferentiated GAPDH-targeted EGFP edited PSCs, or undifferentiated RPL13A-targeted edited PSCs (FIG. 3). For both GAPDH-targeted and RPL13A-targeted EGFP edited PSC lines, we examined one homozygous-targeted line (carrying gene edits in both alleles) and one heterozygous-targeted line (carrying gene edits in one allele). The data demonstrate a high EGFP fluorescence signal from all four edited PSC lines compared to the unedited PSC line. These results indicate that insertion of the EGFP construct at the GAPDH and RPL13A gene loci allowed high levels of transgene expression in edited PSCs.

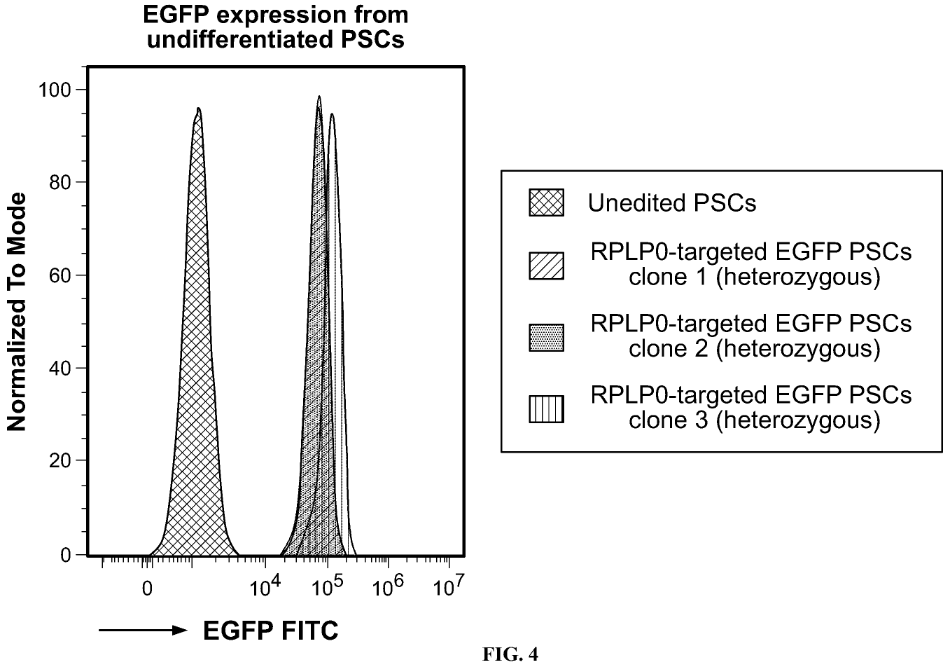

Flow cytometric analysis was performed on either undifferentiated unedited PSCs, or three different undifferentiated clonal PSC lines of RPLP0-targeted EGFP edited PSCs (FIG. 4). All three RPLP0-targeted EGFP PSC lines were heterozygous, carrying gene edits in one allele. The data demonstrate a high EGFP fluorescence signal from all three edited PSC lines compared to the unedited PSC line. These results indicate that insertion of the EGFP construct at the RPLP0 gene locus allowed high levels of transgene expression in edited PSCs.

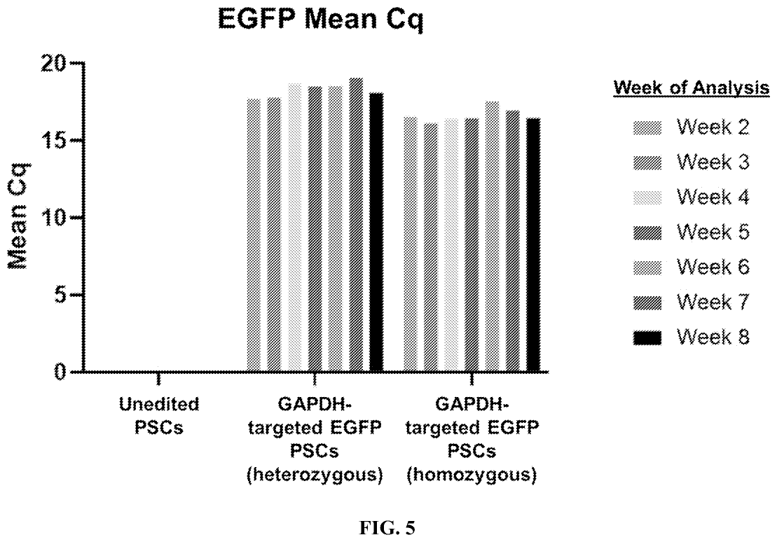

qPCR analysis was performed on RNA collected from unedited PSCs and GAPDH-targeted EGFP edited PSCs on a weekly basis on cell lines cultured for eight weeks (FIG. 5). Cell lines were routinely passaged on average two to three times each week. A mean Cq range between 15 to 20 cycles indicates very high amounts of target RNA and transgene expression. The Cq value is inverse to the amount of target RNA in the sample; the lower the Cq value, the higher the amount of transgene expression. A heterozygous GAPDH-targeted EGFP PSC line (carrying gene edits in one allele) and a homozygous GAPDH-targeted EGFP PSC line (carrying gene edits in both alleles) both demonstrated high transgene expression compared to the unedited PSC line, which did not express EGFP. The homozygous GAPDH-targeted EGFP PSC line displayed slightly lower Cq values than the heterozygous GAPDH-targeted EGFP PSC line, indicating higher transgene expression from the homozygous GAPDH-targeted EGFP PSC line. Both edited PSC lines expressed high levels of EGFP expression each week for up to eight weeks, indicating that high levels of transgene expression were maintained following routine PSC culture for up to eight weeks.

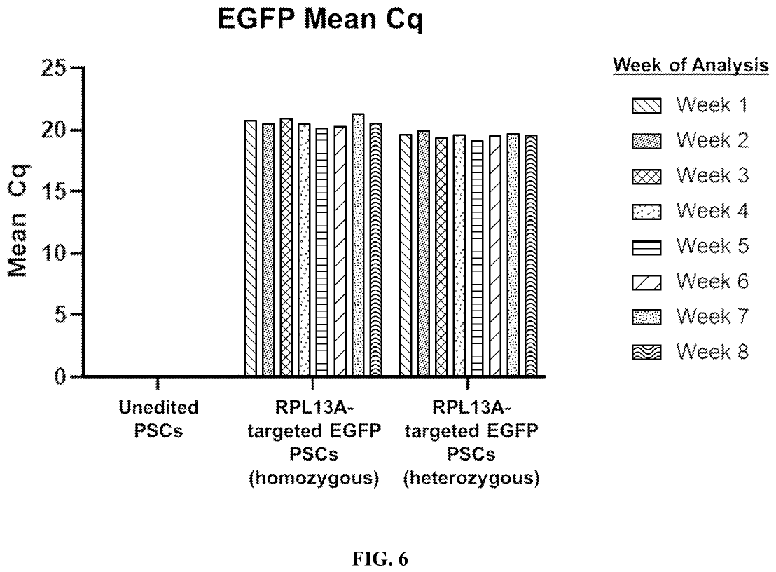

qPCR analysis was performed on RNA collected from unedited PSCs and RPL/3A-targeted EGFP edited PSCs on a weekly basis on cell lines cultured for eight weeks (FIG. 6). Cell lines were routinely passaged on average two to three times each week. A mean Cq range between 15 to 25 cycles indicates very high amounts of target RNA and transgene expression. The Cq value is inverse to the amount of target RNA in the sample; the lower the Cq value, the higher the amount of transgene expression. A heterozygous RPL13A-targeted EGFP PSC line (carrying gene edits in one allele) and a homozygous RPL13A-targeted EGFP PSC line (carrying gene edits in both alleles) both demonstrated high transgene expression compared to the unedited PSC line, which did not express EGFP. The heterozygous RPL/3A-targeted EGFP PSC line displayed slightly lower Cq values than the homozygous RPL/3A-targeted EGFP PSC line, indicating higher transgene expression from the heterozygous RPL13A -targeted EGFP PSC line. Both edited PSC lines expressed high levels of EGFP each week for up to eight weeks, indicating that high levels of transgene expression were maintained following routine PSC culture for up to eight weeks.

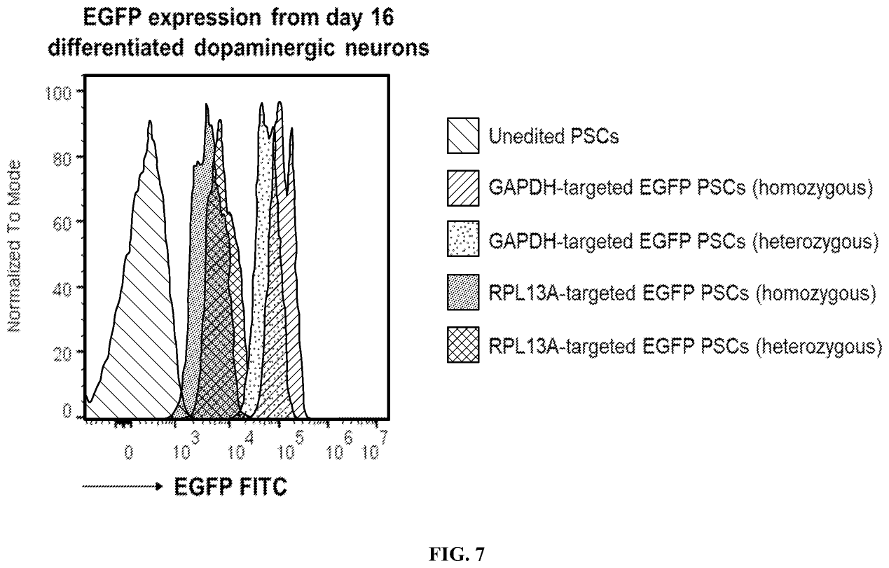

Flow cytometric analysis also was performed on unedited PSCs, GAPDH-targeted EGFP edited PSCs, and RPL13A-targeted EGFP edited PSCs differentiated to day 16 dopaminergic neurons (FIG. 7) (see, e.g., Chambers et al., supra). For both GAPDH-targeted and RPL13A-targeted EGFP edited PSC lines, we examined one homozygous-targeted line (carrying gene edits in both alleles) and one heterozygous-targeted line (carrying gene edits in one allele).

The data demonstrate a high EGFP fluorescence signal from all four edited PSC lines compared to the unedited PSC line following 16 days of differentiation into dopaminergic neurons. These results indicate that insertion of the EGFP construct at the GAPDH and RPL13A gene loci allowed high levels of transgene expression in edited PSCs, and that the high levels of transgene expression were maintained following lineage-directed differentiation of the edited PSCs.

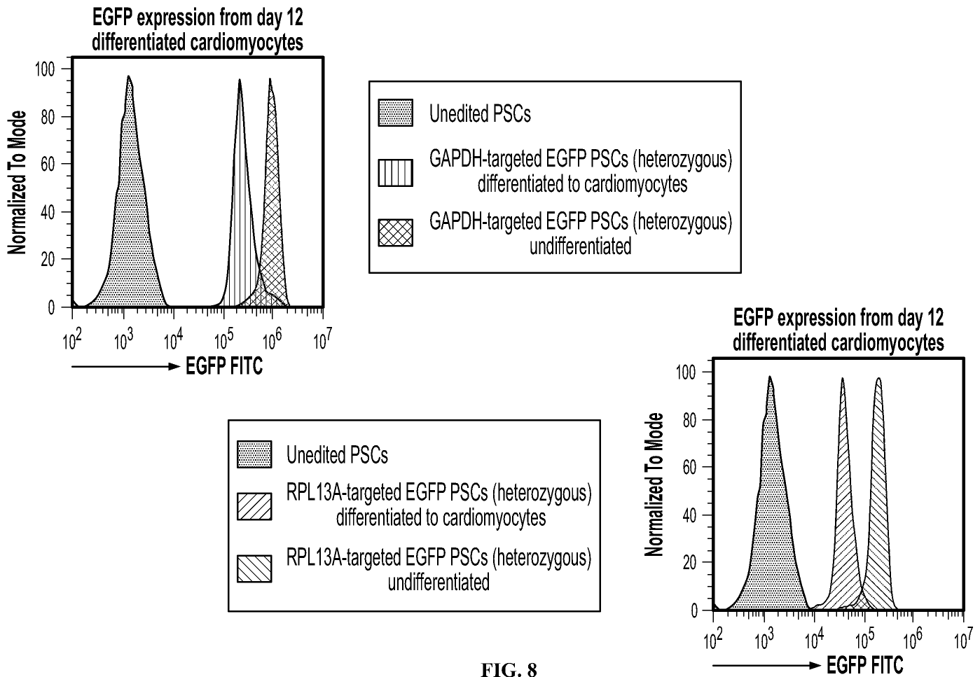

Flow cytometric analysis also was performed on unedited PSCs, a heterozygous GAPDH-targeted EGFP line (carrying gene edits in one allele) differentiated to day 12 cardiomyocytes or undifferentiated, and a heterozygous RPL13A-targeted EGFP line (carrying gene edits in one allele) differentiated to day 12 cardiomyocytes or undifferentiated (FIG. 8) (see, e.g., Lian et al., Nat. Protoc. (2013) 8(1):162-75). The data demonstrate a high EGFP fluorescence from both the GAPDH-targeted EGFP line and the RPL13A-targeted EGFP line compared to the unedited PSC line following 12 days of differentiation into cardiomyocytes. The level of fluorescence of the differentiated edited lines were slightly lower when compared to the undifferentiated edited lines, but remains high. The results indicate that high levels of transgene expression were maintained following cardiomyocyte lineage-directed differentiation of the edited PSCs.

### Example 3: Expression of HLA-G6 at GAPDH and RPL13A Loci in iPSCs

In this study, a construct expressing HLA-G6 was edited into either the GAPDH locus or the RPL13A locus in iPSCs. The HLA-G6 coding sequence is shown below.

(SEQ ID NO: 18)
ATGGTGGTCATGGCGCCCCGAACCCTCTTCCTGCTGCTCTCGGGGGCCCT
GACCCTGACCGAGACCTGGGCGGGCTCCCACTCCATGAGGTATTTCAGCG
CCGCCGTGTCCCGGCCCGGCCGCGGGGAGCCCCGCTTCATCGCCATGGGC
TACGTGGACGACACGCAGTTCGTGCGGTTCGACAGCGACTCGGCGTGTCC
GAGGATGGAGCCGCGGGCGCCGTGGGTGGAGCAGGAGGGGCCGGAGTATT
GGGAAGAGGAGACACGGAACACCAAGGCCCACGCACAGACTGACAGAATG
AACCTGCAGACCCTGCGCGGCTACTACAACCAGAGCGAGGCCAACCCCCC
CAAGACACACGTGACCCACCACCCTGTCTTTGACTATGAGGCCACCCTGA
GGTGCTGGGCCCTGGGCTTCTACCCTGCGGAGATCATACTGACCTGGCAG
CGGGATGGGGAGGACCAGACCCAGGACGTGGAGCTCGTGGAGACCAGGCC
TGCAGGGGATGGAACCTTCCAGAAGTGGGCAGCTGTGGTGGTGCCTTCTG
GAGAGGAGCAGAGATACACGTGCCATGTGCAGCATGAGGGGCTGCCGGAG
CCCCTCATGCTGAGATGGAGTAAGGAGGGAGATGGAGGCATCATGTCTGT
TAGGGAAAGCAGGAGCCTCTCTGAAGACCTTTAA

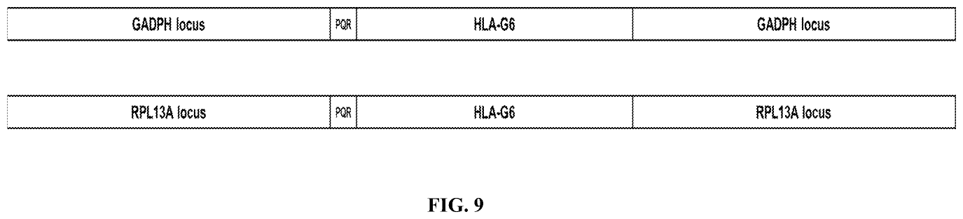

The inserted HLA-G6 transgene was linked in frame to the endogenous housekeeping gene by a PQR sequence as described above (FIG. 9). Each PQR/HLA-G6 insertion construct was also flanked by an 800 bp left homology arm and an 800 bp right homology arm carrying sequences homologous to the endogenous STEL locus (either GAPDH or RPL13A) as described above.

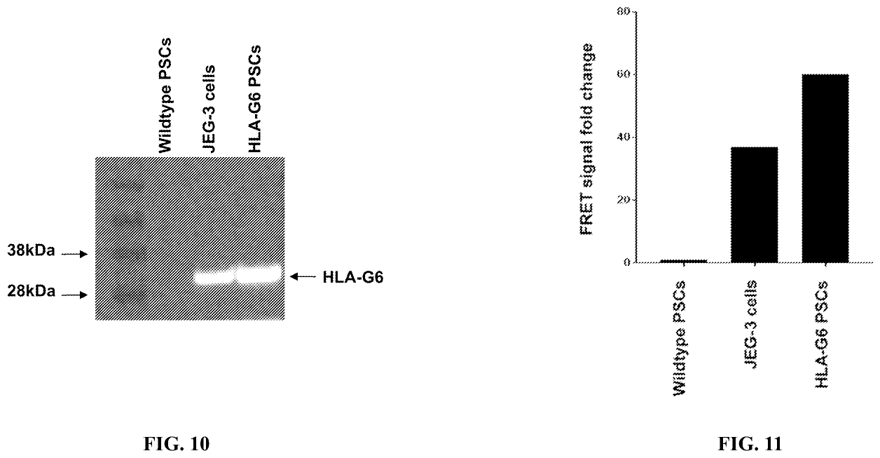

Secretion of HLA-G6 into cell culture media was evaluated by Western blot using an HLA-G5/G6 specific antibody from Thermo Fisher Scientific (clone 5A6G7). Western blot analysis was performed on the cell culture supernatants of unedited wildtype PSCs, control JEG-3 choriocarcinoma cells (derived from human placenta, wherein HLA-G is normally expressed), and the GAPDH-targeted HLA-G6 PSC line (FIG. 10). The primary antibody used was specific to soluble HLA-G isoforms including HLA-G5 and HLA-G6. The predicted protein size of HLA-G6 is approximately 30 kDa. The data demonstrate that HLA-G6 was detected at comparable levels in the cell culture supernatant of the GAPDH-targeted HLA-G6 edited PSC cells and the control JEG-3 cells, but was absent in the cell culture supernatant of unedited PSCs. These results indicate that insertion of the HLA-G6 construct at the GAPDH gene locus allowed the edited PSCs to secrete high levels of HLA-G6.

A fluorescence resonance energy transfer (FRET) detection assay also was performed on the cell culture supernatants of unedited wildtype PSCs, control JEG-3 cells, and the GAPDH-targeted HLA-G6 PSCs (FIG. 11). FRET involves the transfer of energy between two fluorophores, a donor and an acceptor, when in close proximity. The donor molecule was linked to a pan-HLA-G antibody (BD Biosciences; clone 4H84) and the acceptor molecule was linked to an antibody that detects soluble HLA-G isoforms including HLA-G5 and HLA-G6 (Thermo Fisher Scientific; clone 5A6G7). Both antibodies bind secreted HLA-G6 protein, thus enabling FRET to occur between the donor and acceptor molecule. The higher the FRET signal, the greater the amount of protein detected. The data demonstrate a high FRET signal in the cell culture supernatant of control JEG-3 cells and an even higher FRET signal from GAPDH-targeted HLA-G6 edited PSCs, but no signal from unedited PSCs. These results confirm that insertion of the HLA-G6 construct at the GAPDH gene locus allowed the edited PSCs to secrete high levels of HLA-G6.

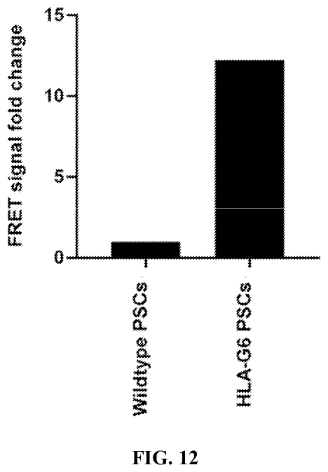

In another study, a FRET detection assay was performed on the cell culture supernatants of unedited PSCs and the RPL13A-targeted HLA-G6 PSC line (FIG. 12). The data demonstrate a high FRET signal in the cell culture supernatant of RPL/3A-targeted HLA-G6 edited PSCs, but little signal from unedited PSCs. These results indicate that insertion of the HLA-G6 construct at the RPL13A gene locus also allowed the edited PSCs to secrete high levels of HLA-G6.

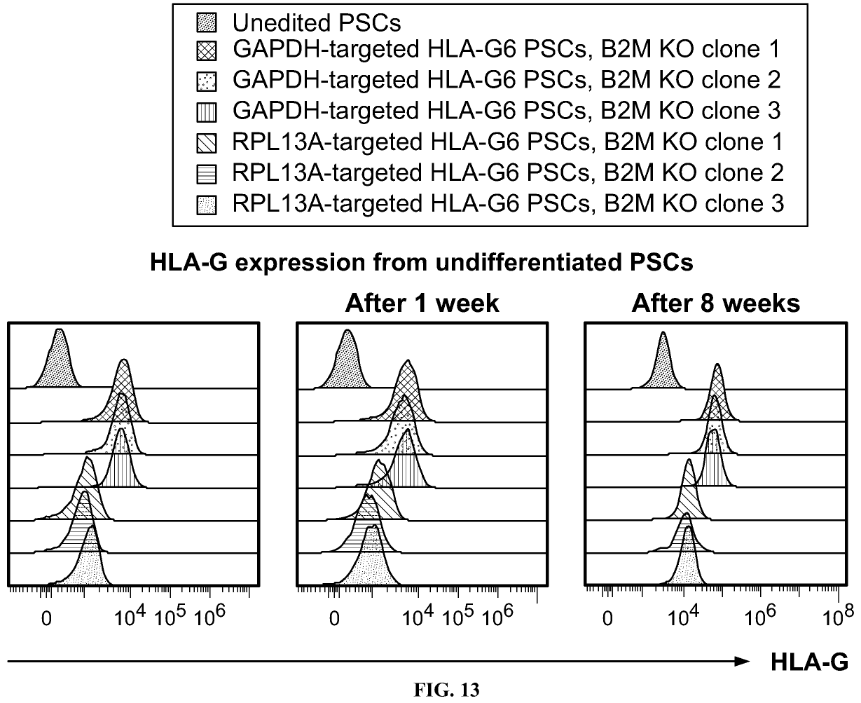

The B2M gene was knocked out using CRISPR/Cas9 gene editing in both the GAPDH-targeted HLA-G6 line and the RPL/3A-targeted HLA-G6 line, and three different B2M knockout (KO) clones were generated for each HLA-G6-edited PSC line. Flow cytometric analysis was performed on all six edited clones using a pan-HLA-G antibody (BD Biosciences; clone 4H84) (FIG. 13). The analysis was repeated following one week of routine PSC culture, and following eight weeks of routine PSC culture. The data demonstrate high HLA-G expression in edited PSC lines compared to the unedited PSC line, and that HLA-G expression is maintained across all edited clonal cell lines for up to eight weeks of routine PSC culture, even after B2M gene knockout. HLA-G expression from GAPDH-targeted PSC lines was higher than RPL13A-targeted PSC lines, indicating higher transgene expression from the GAPDH gene locus.

### Example 4: Expression of Anti-Tau scFv at GAPDH Locus in PSCs

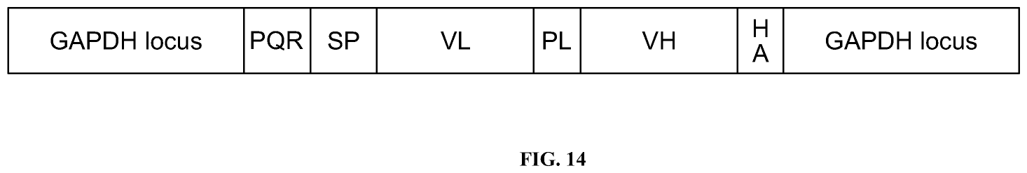

In this study, a construct that expresses a single chain variable fragment (scFv) antibody against human tau (Ising et al., J. Exp. Med. (2017) 214(5):1227-1238) was inserted into the GAPDH locus. The anti-tau scFv insertion construct was comprised of sequences encoding a secretory signal peptide (SP), the light chain variable region (VL) and heavy chain variable region (VH) of the anti-tau antibody HJ8.5 (WO 2016/126993 and WO 2014/008404) linked by a S(GGGGS)₃ (SEQ ID NO:19) peptide linker (PL), and a human influenza hemagglutinin (HA) peptide tag (FIG. 14). The coding sequence for the anti-tau scFv is shown below, where the coding sequence for the secretory signal peptide is boldfaced and underlined, the coding sequence for the VL is italicized, the coding sequence for the peptide linker is boldfaced, the coding sequence for the VH is underlined, and the coding sequence for the HA tag is in boldface and italicized.

(SEQ ID NO: 20)
ATGGATATGAGAGTGCCTGCCCAACTTCTCGGACTGCTGCTGCTTTGGCT
TAGAGGTGCAAGATGCGACATTGTGCTGACACAGTCTCCTGCTTCCTTAG
CTGTATCTCTGGGACAGAGGGCCACCATCTCATGCAGGGCCAGCCAAAGT
GTCAGTACATCTAGATATAGTTATATACACTGGTACCAACAGAAACCAGG
ACAGCCACCCAAACTCCTCATCAAGTATGCATCCAACCTAGAATCTGGGG
TCCCTGCCAGGTTCAGTGGCAGTGGGTCTGGGACAGACTTCACCCTCAAC
ATCCATCCTCTGGAGGAGGAGGATGCTGCAACATATTACTGTCACCACAG
TTGGGAGATTCCGCTCACGTTCGGTGCTGGGACCAAGCTGGAGCTGAAAT
CCGGTGGAGGCGGTTCAGGCGGAGGTGGCTCTGGCGGTGGCGGATCGGAA
GTGAAGGTTGAGGAGTCTGGAGGAGGCTTGGTGCAACCTGGAGGATCCAT
GAAACTCTCCTGTGTTGTCTCTGGATTCACTTTCAGTAACTACTGGGTGA
ACTGGGTCCGCCAGTCTCCAGAGAAGGGGCTTGAGTGGGTTGCTCAAATT
AGATTGAAATCTGATAATTATGCAACACATTATGAGGAGTCTGTGAAAGG
GAGGTTCACCATCTCAAGAGATGATTCCAAAAGTAGTGTCTATCTGCAAA
TGAACAACCTAAGGGCTGAAGACAGTGGAATTTATTACTGCACTAACTGG
GAAGACTACTGGGGCCAAGGCACCACTCTCACAGTCTCCTCATACCCATA
CGATGTTCCAGATTACGCT

A TGA stop codon was incorporated after the transgene coding sequence to permit termination of translation. The expression of the scFv was linked to that of GAPDH by a PQR sequence as described above. Each PQR/anti-tau scFv insertion construct was also flanked by an 800 bp left homology arm and an 800 bp right homology arm as described above.

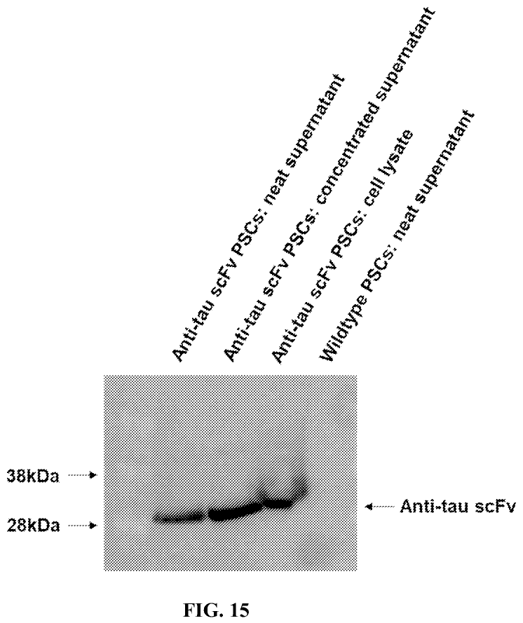

Western blot analysis was performed on the cell culture supernatants of unedited PSCs, the GAPDH-targeted anti-tau scFv PSC line (either neat supernatant or concentrated by anti-HA agarose immunoprecipitation), and the cell lysate of the GAPDH-targeted anti-tau scFv PSC line (FIG. 15). The primary antibody used was an anti-HA monoclonal antibody that recognizes the 9-amino acid sequence YPYDVPDYA (SEQ ID NO:21) derived from the HA peptide tag. The predicted protein size of the anti-tau scFv is approximately 30 kDa.

The data demonstrates that the anti-tau scFv was detected in neat and concentrated cell culture supernatants of the GAPDH-targeted anti-tau scFv edited PSC line, and the cell lysate of the GAPDH-targeted anti-tau scFv edited PSC line, but was absent in the cell culture supernatant of the unedited PSC line. These results indicate that insertion of the anti-tau scFv construct at the GAPDH gene locus allowed the edited PSCs to secrete high levels of the scFv.

### Example 5: Expression of RapaCasp9 Cellular Suicide Switch at GAPDH Locus in PSCs

In this study, two different constructs that together comprise a rapamycin-inducible human Caspase 9-based (RapaCasp9) cellular suicide switch (Stavrou et al., Mol. Ther. (2018) 26(5):1266-76) were inserted into each allele of the GAPDH locus. One RapaCasp9 construct was comprised of sequences encoding the FRB (FKBP12-rapamycin binding) domain of mTOR linked by a SGGGS (SEQ ID NO:22) peptide linker (L1) to a truncated Caspase 9 gene (truncCasp9), which has its CARD domain removed. The other

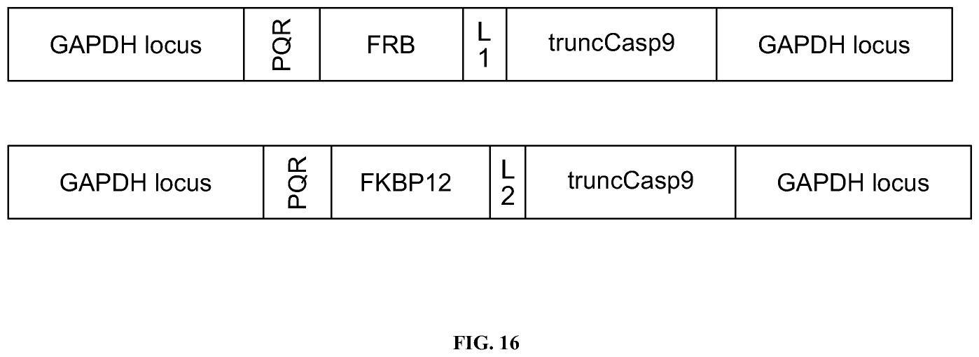

RapaCasp9 construct was comprised of sequences encoding the FKBP12 (FK506-binding protein 12) gene linked by a SGGGS (SEQ ID NO:22) peptide linker (L2) to a truncated Caspase 9 gene (truncCasp9), which has its CARD domain removed (FIG. 16). Addition of the drug rapamycin enables heterodimerization of FRB and FKBP12 which subsequently causes homodimerization of truncated Caspase 9 and induction of apoptosis.

The coding sequence for the FRB-L1-truncCasp9 component of RapaCasp9 is shown below, where the coding sequence for FRB is boldfaced, the coding sequence for the peptide linker (L1) is underlined, and the coding sequence for the truncated Caspase 9 is italicized.

(SEQ ID NO: 23)
ATGGCTTCTAGAATCCTCTGGCATGAGATGTGGCATGAAGGCCTGGAAGA
GGCATCTCGTTTGTACTTTGGGGAAAGGAACGTGAAAGGCATGTTTGAGG
TGCTGGAGCCCTTGCATGCTATGATGGAACGGGGCCCCCAGACTCTGAAG
GAAACATCCTTTAATCAGGCCTATGGTCGAGATTTAATGGAGGCCCAAGA
GTGGTGCAGGAAGTACATGAAATCAGGGAATGTCAAGGACCTCCTCCAAG
CCTGGGACCTCTATTATCATGTGTTCCGACGAATCTCAAAGCTCGAGTAT
AGCGGCGGCGGCAGCGGCGTGGATGGCTTCGGCGACGTGGGAGCCCTGGA
GAGCCTGAGAGGCAACGCCGATCTGGCCTACATCCTGAGCATGGAGCCCT
GTGGCCACTGCCTGATCATCAACAACGTGAACTTCTGCCGGGAGAGCGGC
CTGCGGACCCGGACCGGCAGCAACATCGACTGCGAGAAGCTGAGGAGGCG
CTTCTCCTCCCTGCACTTTATGGTGGAGGTGAAAGGCGATCTGACTGCCA
AGAAAATGGTGCTGGCCCTGCTGGAGCTGGCCCAGCAGGACCACGGAGCC
CTGGATTGCTGTGTGGTGGTGATCCTGTCCCACGGCTGCCAGGCCAGCCA
CCTGCAGTTCCCCGGAGCCGTGTACGGCACCGACGGCTGTCCCGTGTCCG
TGGAGAAGATCGTGAACATCTTCAACGGCACCTCCTGCCCCTCCCTGGGC
GGCAAGCCCAAGCTGTTCTTTATCCAGGCCTGTGGCGGCGAGCAGAAGGA
CCACGGCTTTGAGGTGGCCAGCACCTCCCCCGAGGACGAGAGCCCAGGCA
GCAACCCCGAGCCCGACGCCACCCCCTTCCAGGAGGGCCTGCGCACCTTC
GACCAGCTGGACGCCATCAGCAGCCTGCCCACCCCCAGCGACATCTTCGT
GAGCTACAGCACCTTTCCCGGCTTCGTGAGCTGGCGCGATCCCAAGTCCG
GCTCTTGGTATGTGGAGACCCTGGACGACATCTTTGAGCAGTGGGCTCAT
AGCGAGGACCTGCAGAGCCTGCTGCTGCGCGTGGCCAATGCCGTGAGCGT
GAAGGGCATCTACAAGCAGATGCCAGGCTGCTTCAACTTCCTGCGGAAGA
AGCTGTTCTTCAAGACCAGCGCCTCCTGA

The coding sequence for the FKBP12-L2-truncCasp9 component of RapaCasp9 is shown below, where the coding sequence for FKBP12 is boldfaced, the coding sequence for the peptide linker (L2) is underlined, and the coding sequence for the truncated Caspase 9 is italicized.

(SEQ ID NO: 24)
ATGCTGGAGGGCGTGCAGGTGGAGACCATCAGCCCAGGCGACGGCAGAAC
CTTCCCCAAGAGAGGCCAGACCTGCGTGGTGCACTATACCGGCATGCTGG
AGGACGGCAAGAAGTTCGACAGCAGCCGCGACCGCAATAAGCCCTTCAAG
TTCATGCTGGGCAAGCAGGAGGTGATCAGAGGCTGGGAGGAGGGCGTGGC
CCAGATGAGCGTGGGCCAGAGAGCCAAGCTGACCATCAGCCCCGACTACG
CCTATGGCGCCACCGGCCACCCCGGCATCATCCCACCCCACGCCACCCTG
GTGTTTGATGTGGAGCTGCTGAAGCTGGAGTCCGGAGGCGGCTCCGGCGT
GGATGGCTTCGGCGACGTGGGAGCCCTGGAGAGCCTGAGAGGCAACGCCG
ATCTGGCCTACATCCTGAGCATGGAGCCCTGTGGCCACTGCCTGATCATC
AACAACGTGAACTTCTGCCGGGAGAGCGGCCTGCGGACCCGGACCGGCAG
CAACATCGACTGCGAGAAGCTGAGGAGGCGCTTCTCCTCCCTGCACTTTA
TGGTGGAGGTGAAAGGCGATCTGACTGCCAAGAAAATGGTGCTGGCCCTG
CTGGAGCTGGCCCAGCAGGACCACGGAGCCCTGGATTGCTGTGTGGTGGT
GATCCTGTCCCACGGCTGCCAGGCCAGCCACCTGCAGTTCCCCGGAGCCG
TGTACGGCACCGACGGCTGTCCCGTGTCCGTGGAGAAGATCGTGAACATC
TTCAACGGCACCTCCTGCCCCTCCCTGGGCGGCAAGCCCAAGCTGTTCTT
TATCCAGGCCTGTGGCGGCGAGCAGAAGGACCACGGCTTTGAGGTGGCCA
GCACCTCCCCCGAGGACGAGAGCCCAGGCAGCAACCCCGAGCCCGACGCC
ACCCCCTTCCAGGAGGGCCTGCGCACCTTCGACCAGCTGGACGCCATCAG
CAGCCTGCCCACCCCCAGCGACATCTTCGTGAGCTACAGCACCTTTCCCG
GCTTCGTGAGCTGGCGCGATCCCAAGTCCGGCTCTTGGTATGTGGAGACC
CTGGACGACATCTTTGAGCAGTGGGCTCATAGCGAGGACCTGCAGAGCCT
GCTGCTGCGCGTGGCCAATGCCGTGAGCGTGAAGGGCATCTACAAGCAGA
TGCCAGGCTGCTTCAACTTCCTGCGGAAGAAGCTGTTCTTCAAGACCAGC
GCCTCCTGA

A TGA stop codon was incorporated after each transgene coding sequence to permit termination of translation. The expression of both the FRB-L1-truncCasp9 and FKBP12-L2-truncCasp9 components of RapaCasp9 was linked to that of GAPDH by a PQR sequence as described above. Each PQR/RapaCasp9 construct was also flanked by an 800 bp left homology arm and an 800 bp right homology arm as described above.

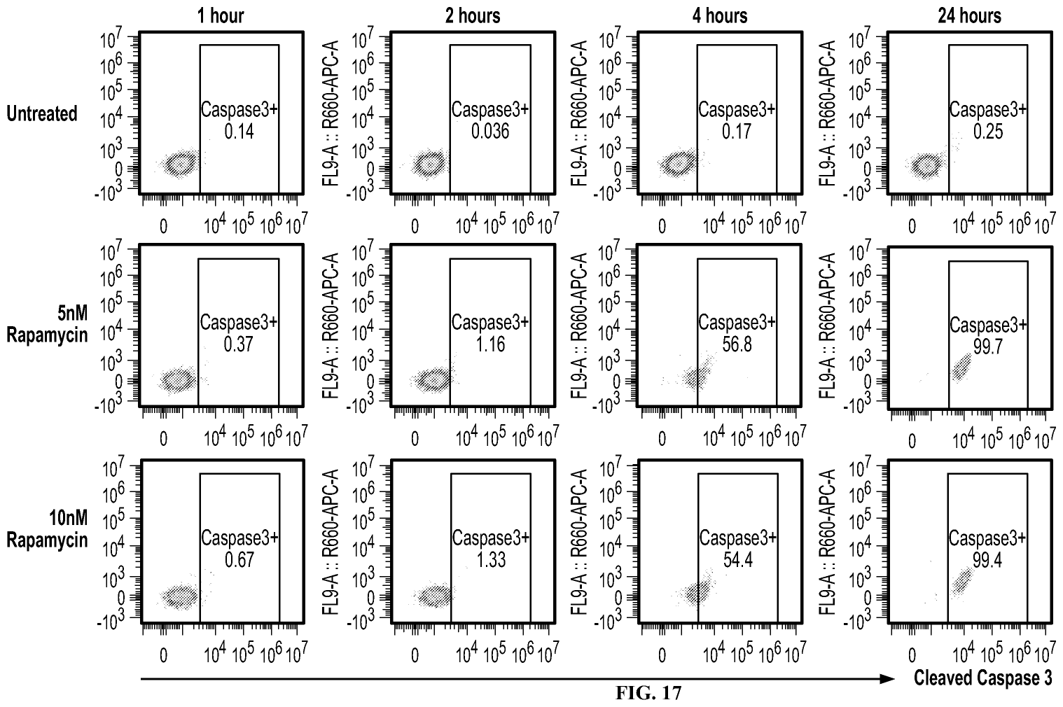

A GAPDH-targeted RapaCasp9 PSC line was treated with either 5nM or 10 nM of rapamycin for 1, 2, 4 or 24 hours, and cells were harvested for flow cytometric analysis after each timepoint (FIG. 17). The primary antibody used was an anti-human/mouse cleaved caspase-3 conjugated to an Alexa Fluor® 488 secondary antibody. The primary antibody detects human and mouse Caspase 3 cleaved at Asp175. Caspase 3 is an executioner caspase that functions downstream of the initiator caspase, Caspase 9, in the apoptotic cascade. Human Procaspase 3 is normally an inactive homodimer. Upon induction of apoptosis through either cell stress or activation, it undergoes proteolysis into cleaved Caspase 3 subunits. The data demonstrates after treatment of the GAPDH-targeted RapaCasp9 PSC line with either 5nM or 10 nM of rapamycin, cleaved Caspase 3 staining is readily detectable after 4 hours of treatment, and almost all cells (>99%) stain for cleave Caspase 3 after 24 hours of treatment. There was negligible detection of cleaved Caspase 3 for edited PSCs not treated with rapamycin. These results indicate that biallelic insertion of FRB-L1-truncCasp9 and FKBP12-L2-truncCasp9 RapaCasp9 constructs at the GAPDH gene locus allowed the edited PSCs to under apoptosis upon induction with rapamycin.

### Example 6: Expression of PD-L1 and CD47 Immunoregulatory Molecules at GAPDH Locus in PSCs

In this study, two different constructs that each contain both an immuno-modulatory molecule and the HSV-TK.007 (herpes simplex thymidine kinase) cellular suicide switch were inserted into each allele of the GAPDH locus. The PD-L1-based construct was comprised of the coding sequence of PD-L1 (programmed death ligand 1) linked via an internal ribosome entry site (IRES) sequence to the coding sequence for HSV-TK.007, which was linked via a P2A sequence to the coding sequence for puroR (puromycin resistance gene). The CD47-based construct was comprised of the coding sequence of CD47 linked via an IRES sequence to the coding sequence for HSV-TK.007. Upon addition of ganciclovir, cells containing these constructs convert ganciclovir to a toxic nucleotide analog which causes DNA replication failure and cell death in actively proliferating cells.

The coding sequence for the PD-L1-based construct is shown below, where the coding sequence for PD-L1 is boldfaced, the coding sequence for the IRES is underlined, the coding sequence for the HSV-TK.007 is italicized, the coding sequence for the P2A (including a GSG linker) is boldfaced and underlined, and the coding sequence for puroR is in regular script.

(SEQ ID NO: 25)
ATGAGGATATTTGCTGTCTTTATATTCATGACCTACTGGCATTTGCTGAA
CGCATTTACTGTCACGGTTCCCAAGGACCTATATGTGGTAGAGTATGGTA
GCAATATGACAATTGAATGCAAATTCCCAGTAGAAAAACAATTAGACCTG
GCTGCACTAATTGTCTATTGGGAAATGGAGGATAAGAACATTATTCAATT
TGTGCATGGAGAGGAAGACCTGAAGGTTCAGCATAGTAGCTACAGACAGA
GGGCCCGGCTGTTGAAGGACCAGCTCTCCCTGGGAAATGCTGCACTTCAG
ATCACAGATGTGAAATTGCAGGATGCAGGGGTGTACCGCTGCATGATCAG
CTATGGTGGTGCCGACTACAAGCGAATTACTGTGAAAGTCAATGCCCCAT
ACAACAAAATCAACCAAAGAATTTTGGTTGTGGATCCAGTCACCTCTGAA
CATGAACTGACATGTCAGGCTGAGGGCTACCCCAAGGCCGAAGTCATCTG
GACAAGCAGTGACCATCAAGTCCTGAGTGGTAAGACCACCACCACCAATT
CCAAGAGAGAGGAGAAGCTTTTCAATGTGACCAGCACACTGAGAATCAAC
ACAACAACTAATGAGATTTTCTACTGCACTTTTAGGAGATTAGATCCTGA
GGAAAACCATACAGCTGAATTGGTCATCCCAGAACTACCTCTGGCACATC
CTCCAAATGAAAGGACTCACTTGGTAATTCTGGGAGCCATCTTATTATGC
CTTGGTGTAGCACTGACATTCATCTTCCGTTTAAGAAAAGGGAGAATGAT
GGATGTGAAAAAATGTGGCATCCAAGATACAAACTCAAAGAAGCAAAGTG
ATACACATTTGGAGGAGACGTAACCCCTCTCCCTCCCCCCCCCCTAACGT
TACTGGCCGAAGCCGCTTGGAATAAGGCCGGTGTGCGTTTGTCTATATGT
TATTTTCCACCATATTGCCGTCTTTTGGCAATGTGAGGGCCCGGAAACCT
GGCCCTGTCTTCTTGACGAGCATTCCTAGGGGTCTTTCCCCTCTCGCCAA
AGGAATGCAAGGTCTGTTGAATGTCGTGAAGGAAGCAGTTCCTCTGGAAG
CTTCTTGAAGACAAACAACGTCTGTAGCGACCCTTTGCAGGCAGCGGAAC
CCCCCACCTGGCGACAGGTGCCTCTGCGGCCAAAAGCCACGTGTATAAGA
TACACCTGCAAAGGCGGCACAACCCCAGTGCCACGTTGTGAGTTGGATAG
TTGTGGAAAGAGTCAAATGGCTCTCCTCAAGCGTATTCAACAAGGGGCTG
AAGGATGCCCAGAAGGTACCCCATTGTATGGGATCTGATCTGGGGCCTCG
GTGCACATGCTTTACATGTGTTTAGTCGAGGTTAAAAAACGTCTAGGCCC
CCCGAACCACGGGGACGTGGTTTTCCTTTGAAAAACACGATGATAATATG
GCCACAACCATGGCCAGCTACCCCTGTCACCAGCACGCCAGCGCCTTCGA
CCAGGCCGCCAGAAGCAGGGGCCACAGCAACCGGCGGACCGCCTTAAGAC
CCAGGCGGCAGCAGGAAGCCACCGAAGTCCGGCTGGAACAGAAGATGCCC
ACCCTGCTGCGGGTGTACATCGACGGCCCCCACGGCATGGGCAAGACCAC
CACCACCCAGCTGCTGGTGGCCCTGGGCAGCCGGGACGACATCGTGTACG
TGCCCGAGCCCATGACCTACTGGCAGGTGCTGGGCGCCAGCGAGACCATC
GCCAACATCTACACCACACAGCACAGGCTGGACCAGGGCGAGATCTCTGC
CGGCGACGCCGCCGTGGTGATGACCAGCGCCCAGATCACAATGGGCATGC
CCTACGCCGTGACCGACGCCGTGCTGGCCCCTCACGTGGGCGGCGAGGCC
GGCTCTAGCCACGCCCCTCCCCCTGCCCTGACCCTGATCTTCGACCGGCA
CCCCATCGCCCACCTGCTGTGCTACCCTGCCGCCAGATACCTGATGGGCA
GCATGACCCCCCAGGCCGTGCTGGCCTTCGTGGCCCTGATCCCCCCCACC
CTGCCCGGCACCAACATCGTGCTGGGAGCCCTGCCCGAGGACCGGCACAT
CGACCGGCTGGCCAAGCGGCAGAGACCCGGCGAGCGGCTGGACCTGGCCA
TGCTGGCCGCCATCCGGCGGGTGTACGGCCTGCTGGCCAACACCGTGAGA
TACCTGCAGGGCGGAGGGTCTTGGTGGGAGGACTGGGGCCAGCTGTCCGG
CACCGCCGTGCCACCTCAGGGCGCCGAGCCCCAGAGCAATGCCGGCCCTC
GGCCCCACATCGGCGACACCCTGTTTACCCTGTTCAGAGCCCCCGAGCTG
CTGGCCCCCAACGGCGACCTGTACAACGTGTTCGCCTGGGCCCTGGACGT
GCTGGCCAAGAGGCTGCGGCCCATGCACGTGTTCATCCTGGACTACGACC
AGAGCCCTGCCGGCTGCAGGGACGCCCTGCTGCAGCTGACCAGCGGCATG
GTGCAGACCCACGTGACCACCCCCGGCAGCATCCCCACCATCTGCGACCT
GGCCCGGACCTTCGCCCGGGAGATGGGCGAGGCCAACGGAAGCGGAGCTA
CTAACTTCAGCCTGCTGAAGCAGGCTGGCGACGTGGAGGAGAACCCTGGA
CCTATGACCGAGTACAAGCCCACGGTGCGCCTCGCCACCCGCGACGACGT
CCCCCGGGCCGTACGCACCCTCGCCGCCGCGTTCGCCGACTACCCCGCCA
CGCGCCACACCGTCGACCCGGACCGCCACATCGAGCGGGTCACCGAGCTG
CAAGAACTCTTCCTCACGCGCGTCGGGCTCGACATCGGCAAGGTGTGGGT
CGCGGACGACGGCGCCGCGGTGGCGGTCTGGACCACGCCGGAGAGCGTCG
AAGCGGGGGCGGTGTTCGCCGAGATCGGCCCGCGCATGGCCGAGTTGAGC
GGTTCCCGGCTGGCCGCGCAGCAACAGATGGAAGGCCTCCTGGCGCCGCA
CCGGCCCAAGGAGCCCGCGTGGTTCCTGGCCACCGTCGGCGTCTCGCCCG
ACCACCAGGGCAAGGGTCTGGGCAGCGCCGTCGTGCTCCCCGGAGTGGAG
GCGGCCGAGCGCGCCGGGGTGCCCGCCTTCCTGGAGACCTCCGCGCCCCG
CAACCTCCCCTTCTACGAGCGGCTCGGCTTCACCGTCACCGCCGACGTCG
AGGTGCCCGAAGGACCGCGCACCTGGTGCATGACCCGCAAGCCCGGTGCC
TGA

The coding sequence for the CD47-based construct is shown below, where the coding sequence for CD47 is boldfaced, the coding sequence for the IRES is underlined, the coding effluence for the HSV-TK 007 is italicized.

(SEQ ID NO: 26)
ATGTGGCCCCTGGTAGCGGCGCTGTTGCTGGGCTCGGCGTGCTGCGGATC
AGCTCAGCTACTATTTAATAAAACAAAATCTGTAGAATTCACGTTTTGTA
ATGACACTGTCGTCATTCCATGCTTTGTTACTAATATGGAGGCACAAAAC
ACTACTGAAGTATACGTAAAGTGGAAATTTAAAGGAAGAGATATTTACAC
CTTTGATGGAGCTCTAAACAAGTCCACTGTCCCCACTGACTTTAGTAGTG
CAAAAATTGAAGTCTCACAATTACTAAAAGGAGATGCCTCTTTGAAGATG
GATAAGAGTGATGCTGTCTCACACACAGGAAACTACACTTGTGAAGTAAC
AGAATTAACCAGAGAAGGTGAAACGATCATCGAGCTAAAATATCGTGTTG
TTTCATGGTTTTCTCCAAATGAAAATATTCTTATTGTTATTTTCCCAATT
TTTGCTATACTCCTGTTCTGGGGACAGTTTGGTATTAAAACACTTAAATA
TAGATCCGGTGGTATGGATGAGAAAACAATTGCTTTACTTGTTGCTGGAC
TAGTGATCACTGTCATTGTCATTGTTGGAGCCATTCTTTTCGTCCCAGGT
GAATATTCATTAAAGAATGCTACTGGCCTTGGTTTAATTGTGACTTCTAC
AGGGATATTAATATTACTTCACTACTATGTGTTTAGTACAGCGATTGGAT
TAACCTCCTTCGTCATTGCCATATTGGTTATTCAGGTGATAGCCTATATC
CTCGCTGTGGTTGGACTGAGTCTCTGTATTGCGGCGTGTATACCAATGCA
TGGCCCTCTTCTGATTTCAGGTTTGAGTATCTTAGCTCTAGCACAATTAC
TTGGACTAGTTTATATGAAATTTGTGGAATAACCCCTCTCCCTCCCCCCC
CCCTAACGTTACTGGCCGAAGCCGCTTGGAATAAGGCCGGTGTGCGTTTG
TCTATATGTTATTTTCCACCATATTGCCGTCTTTTGGCAATGTGAGGGCC
CGGAAACCTGGCCCTGTCTTCTTGACGAGCATTCCTAGGGGTCTTTCCCC
TCTCGCCAAAGGAATGCAAGGTCTGTTGAATGTCGTGAAGGAAGCAGTTC
CTCTGGAAGCTTCTTGAAGACAAACAACGTCTGTAGCGACCCTTTGCAGG
CAGCGGAACCCCCCACCTGGCGACAGGTGCCTCTGCGGCCAAAAGCCACG
TGTATAAGATACACCTGCAAAGGCGGCACAACCCCAGTGCCACGTTGTGA
GTTGGATAGTTGTGGAAAGAGTCAAATGGCTCTCCTCAAGCGTATTCAAC
AAGGGGCTGAAGGATGCCCAGAAGGTACCCCATTGTATGGGATCTGATCT
GGGGCCTCGGTGCACATGCTTTACATGTGTTTAGTCGAGGTTAAAAAACG
TCTAGGCCCCCCGAACCACGGGGACGTGGTTTTCCTTTGAAAAACACGAT
GATAATATGGCCACAACCATGGCCAGCTACCCCTGTCACCAGCACGCCAG
CGCCTTCGACCAGGCCGCCAGAAGCAGGGGCCACAGCAACCGGCGGACCG
CCTTAAGACCCAGGCGGCAGCAGGAAGCCACCGAAGTCCGGCTGGAACAG
AAGATGCCCACCCTGCTGCGGGTGTACATCGACGGCCCCCACGGCATGGG
CAAGACCACCACCACCCAGCTGCTGGTGGCCCTGGGCAGCCGGGACGACA
TCGTGTACGTGCCCGAGCCCATGACCTACTGGCAGGTGCTGGGCGCCAGC
GAGACCATCGCCAACATCTACACCACACAGCACAGGCTGGACCAGGGCGA
GATCTCTGCCGGCGACGCCGCCGTGGTGATGACCAGCGCCCAGATCACAA
TGGGCATGCCCTACGCCGTGACCGACGCCGTGCTGGCCCCTCACGTGGGC
GGCGAGGCCGGCTCTAGCCACGCCCCTCCCCCTGCCCTGACCCTGATCTT
CGACCGGCACCCCATCGCCCACCTGCTGTGCTACCCTGCCGCCAGATACC
TGATGGGCAGCATGACCCCCCAGGCCGTGCTGGCCTTCGTGGCCCTGATC
CCCCCCACCCTGCCCGGCACCAACATCGTGCTGGGAGCCCTGCCCGAGGA
CCGGCACATCGACCGGCTGGCCAAGCGGCAGAGACCCGGCGAGCGGCTGG
ACCTGGCCATGCTGGCCGCCATCCGGCGGGTGTACGGCCTGCTGGCCAAC
ACCGTGAGATACCTGCAGGGCGGAGGGTCTTGGTGGGAGGACTGGGGCCA
GCTGTCCGGCACCGCCGTGCCACCTCAGGGCGCCGAGCCCCAGAGCAATG
CCGGCCCTCGGCCCCACATCGGCGACACCCTGTTTACCCTGTTCAGAGCC
CCCGAGCTGCTGGCCCCCAACGGCGACCTGTACAACGTGTTCGCCTGGGC
CCTGGACGTGCTGGCCAAGAGGCTGCGGCCCATGCACGTGTTCATCCTGG
ACTACGACCAGAGCCCTGCCGGCTGCAGGGACGCCCTGCTGCAGCTGACC
AGCGGCATGGTGCAGACCCACGTGACCACCCCCGGCAGCATCCCCACCAT
CTGCGACCTGGCCCGGACCTTCGCCCGGGAGATGGGCGAGGCCAACTAA

A stop codon was incorporated after each transgene coding sequence to permit termination of translation. The expression of the PD-L1-based construct was linked to that of GAPDH by a PQR sequence as described above, and flanked by an 800 bp left homology arm and an 800 bp right homology arm as described above. The expression of the CD47-based construct was linked to that of GAPDH by a P2A sequence, where the GSG linker is in boldfaced in the sequence below, and flanked by an 800 bp left homology arm and an 800 bp right homology arm as described above:

(SEQ ID NO: 27)
GGAAGCGGAGCTACTAACTTCAGCCTGCTGAAGCAGGCTGGCGACGTGGA
GGAGAACCCTGGACCT

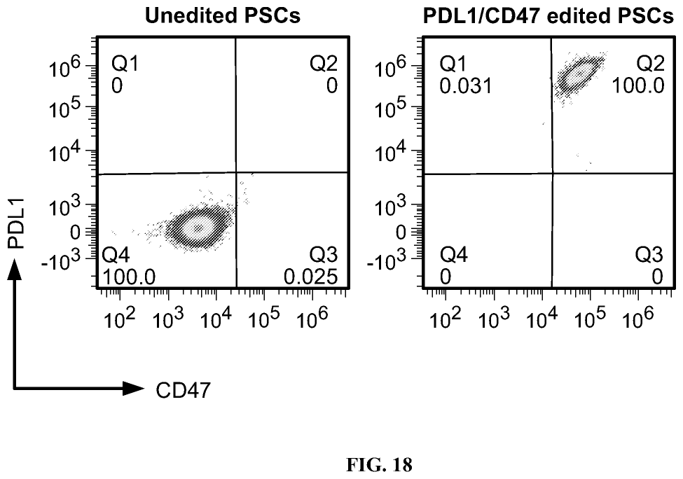

Flow cytometric analysis was performed on unedited PSCs, or GAPDH-targeted PSCs that contained one allele edited with the PD-L1-based construct, and the other allele edited with the CD47-based construct (FIG. 18). The data demonstrate detection of dual PD-L1 and CD47 co-staining in the GAPDH-targeted PSCs but no staining in the unedited PSCs, indicating that biallelic insertion of a PD-L1-based construct and a CD47-based construct at the GAPDH locus allowed the edited PSCs to express PD-L1 and CD47.

### Example 7: Expression of CSF1 at GAPDH Locus in PSCs

In this study, a construct containing the coding sequence for CSF1 (colony stimulating factor 1) was inserted into either one or both alleles of the GAPDH locus. CSF1 is a cytokine that controls the survival, differentiation, and function of macrophages. The coding sequence for CSF1 is shown below.

(SEQ ID NO: 28)
ATGACCGCGCCGGGCGCCGCCGGGCGCTGCCCTCCCACGACATGGCTGGG
CTCCCTGCTGTTGTTGGTCTGTCTCCTGGCGAGCAGGAGTATCACCGAGG
AGGTGTCGGAGTACTGTAGCCACATGATTGGGAGTGGACACCTGCAGTCT
CTGCAGCGGCTGATTGACAGTCAGATGGAGACCTCGTGCCAAATTACATT
TGAGTTTGTAGACCAGGAACAGTTGAAAGATCCAGTGTGCTACCTTAAGA
AGGCATTTCTCCTGGTACAAGACATAATGGAGGACACCATGCGCTTCAGA
GATAACACCCCCAATGCCATCGCCATTGTGCAGCTGCAGGAACTCTCTTT
GAGGCTGAAGAGCTGCTTCACCAAGGATTATGAAGAGCATGACAAGGCCT
GCGTCCGAACTTTCTATGAGACACCTCTCCAGTTGCTGGAGAAGGTCAAG
AATGTCTTTAATGAAACAAAGAATCTCCTTGACAAGGACTGGAATATTTT
CAGCAAGAACTGCAACAACAGCTTTGCTGAATGCTCCAGCCAAGATGTGG
TGACCAAGCCTGATTGCAACTGCCTGTACCCCAAAGCCATCCCTAGCAGT
GACCCGGCCTCTGTCTCCCCTCATCAGCCCCTCGCCCCCTCCATGGCCCC
TGTGGCTGGCTTGACCTGGGAGGACTCTGAGGGAACTGAGGGCAGCTCCC
TCTTGCCTGGTGAGCAGCCCCTGCACACAGTGGATCCAGGCAGTGCCAAG
CAGCGGCCACCCAGGAGCACCTGCCAGAGCTTTGAGCCGCCAGAGACCCC
AGTTGTCAAGGACAGCACCATCGGTGGCTCACCACAGCCTCGCCCCTCTG
TCGGGGCCTTCAACCCCGGGATGGAGGATATTCTTGACTCTGCAATGGGC
ACTAATTGGGTCCCAGAAGAAGCCTCTGGAGAGGCCAGTGAGATTCCCGT
ACCCCAAGGGACAGAGCTTTCCCCCTCCAGGCCAGGAGGGGGCAGCATGC
AGACAGAGCCCGCCAGACCCAGCAACTTCCTCTCAGCATCTTCTCCACTC
CCTGCATCAGCAAAGGGCCAACAGCCGGCAGATGTAACTGGTACCGCCTT
GCCCAGGGTGGGCCCCGTGAGGCCCACTGGCCAGGACTGGAATCACACCC
CCCAGAAGACAGACCATCCATCTGCCCTGCTCAGAGACCCCCCGGAGCCA
GGCTCTCCCAGGATCTCATCACTGCGCCCCCAGGGCCTCAGCAACCCCTC
CACCCTCTCTGCTCAGCCACAGCTTTCCAGAAGCCACTCCTCGGGCAGCG
TGCTGCCCCTTGGGGAGCTGGAGGGCAGGAGGAGCACCAGGGATCGGAGG
AGCCCCGCAGAGCCAGAAGGAGGACCAGCAAGTGAAGGGGCAGCCAGGCC
CCTGCCCCGTTTTAACTCCGTTCCTTTGACTGACACAGGCCATGAGAGGC
AGTCCGAGGGATCCTTCAGCCCGCAGCTCCAGGAGTCTGTCTTCCACCTG
CTGGTGCCCAGTGTCATCCTGGTCTTGCTGGCCGTCGGAGGCCTCTTGTT
CTACAGGTGGAGGCGGCGGAGCCATCAAGAGCCTCAGAGAGCGGATTCTC
CCTTGGAGCAACCAGAGGGCAGCCCCCTGACTCAGGATGACAGACAGGTG
GAACTGCCAGTGTAG

A TAG stop codon was incorporated after the transgene coding sequence to permit termination of translation. The expression of CSF1 was linked to that of GAPDH by a PQR sequence as described above. Each PQR/CSF1 insertion construct was also flanked by an 800 bp left homology arm and an 800 bp right homology arm as described above.

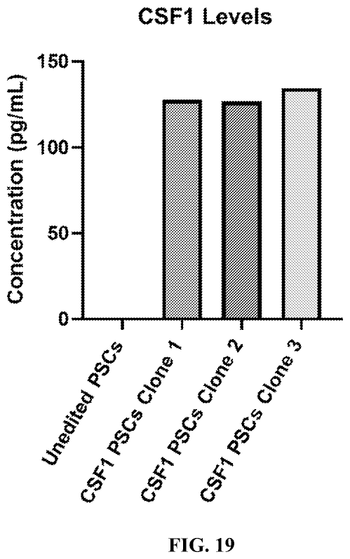

An ELISA immunoassay was performed on the cell culture supernatants of unedited PSCs and three different GAPDH-targeted CSF1 PSC lines (FIG. 19). The data demonstrate that secreted CSF1 was detected in the cell culture supernatants of all three GAPDH-targeted CSF1 edited PSC lines, but was absent in the cell culture supernatant of the unedited PSC line. These results indicate that insertion of the CSF1 construct at the GAPDH locus allowed the edited PSCs to secrete readily detectable levels of CSF1.

### Example 8: Transgene Silencing of PD-L1 at AAVS1 Locus in Differentiated PSCs

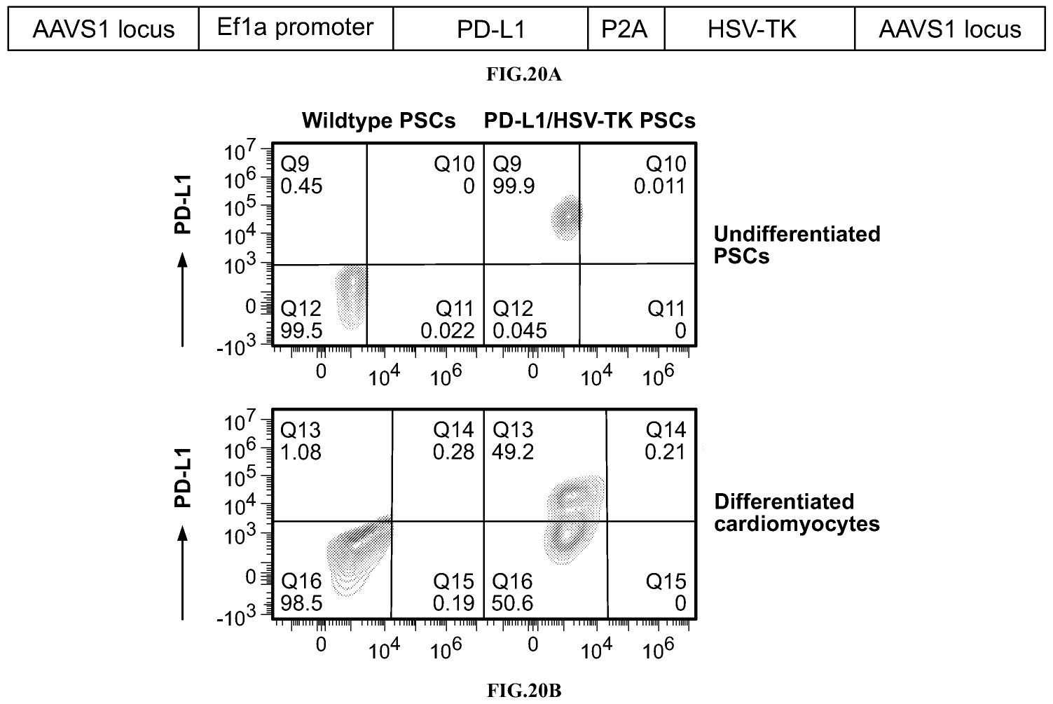

In this study, we used CRISPR-Cas9 gene editing to insert a construct that expresses PD-L1 at the AAVS1 safe harbor locus (FIG. 20A). The insertion construct included an external EF1a promoter to drive expression of the transgene construct. Additionally, HSV-TK, a suicide gene that can be induced to eliminate proliferating cells via small molecule treatment, was linked to PD-L1 by a P2A sequence, which permits bicistronic expression of both PD-L1 and HSV-TK upon cleavage of P2A. The insertion construct was also flanked by left and right homology arms carrying sequences homologous to the endogenous AAVS1 locus to enable integration of the construct at its intended targeting site. Flow cytometric analysis was performed on either undifferentiated wildtype PSCs, or undifferentiated AA VS1-targeted PD-L1/HSV-TK edited PSCs (FIG. 20B). Cells were stained with an anti-PD-L1 primary antibody.

The data demonstrate that a majority of PSCs carrying the PD-L1/HSV-TK edit (99.9%) express PD-L1 by flow cytometry, whereas no wildtype PSCs express PD-Ll. Both PSC lines were subsequently differentiated into cardiomyocytes and analyzed by flow cytometry following staining with an anti-PD-L1 primary antibody. The data demonstrate that only 49% of PSCs carrying the PD-L1/HSV-TK edit express PD-L1 by flow cytometry. These results indicate that insertion of the PD-L1/HSV-TK construct at the AAVS1 locus leads to transgene silencing of PD-L1 expression upon lineage-directed differentiation of PSCs into cardiomyocytes. Similar transgene silencing was observed at the B2M locus.

In conclusion, the above data show that the GAPDH, RPL13A and RPLP0 gene loci allow sustained, high levels of expression of a variety of transgenes integrated therein. Our data also indicate that a transgene (e.g., one encoding PD-L1) integrated at the commonly used AAVS1 and B2M loci lost its expression in the edited cells once the cells differentiated from PSCs to cardiomyocytes.

1. A genetically modified mammalian cell comprising a transgene at a sustained transgene expression locus (STEL) in the genome, wherein the transgene is expressed at a detectable level, optionally wherein the mammalian cell is a human cell.

2. The genetically modified mammalian cell of claim 1, wherein the expression level of the transgene does not change more than 40%, more than 30%, more than 20%, or more than 10% (i) over five or more, ten or more, or 15 or more passages, or (ii) as the cell state changes, wherein the cell state is optionally state of pluripotency and/or differentiation.

3. The genetically modified mammalian cell of claim 1, wherein the STEL is selected from the gene loci listed in Table 1.

4. The genetically modified mammalian cell of claim 3, wherein the STEL is a gene locus having a mean normalized expression of more than 3.30, more than 3.50, more than 3.75, more than 4.00, more than 4.10, more than 4.20, more than 4.30, more than 4.50, more than 4.60, more than 4.70 as set forth in Table 1.

5. The genetically modified mammalian cell of claim 3, wherein the STEL is at a gene that encodes a protein involved with one or more of: ribonucleoprotein complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and protein binding.

6. The genetically modified mammalian cell of claim 3, wherein the STEL is a gene encoding a ribosomal protein, optionally (i) an RPL gene selected from RPL13A, RPLP0, RPL10, RPL13, RPS18, RPL3, RPLP1, RPL15, RPL41, RPL11, RPL32, RPL18A, RPL19, RPL28, RPL29, RPL9, RPL8, RPL6, RPL18, RPL7, RPL7A, RPL21, RPL37A, RPL12, RPL5, RPL34, RPL35A, RPL30, RPL24, RPL39, RPL37, RPL14, RPL27A, RPLP2, RPL23A, RPL26, RPL36, RPL35, RPL23, RPL4, and RPL22; or (ii) a RPS gene selected from RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS16, RPS25, RPS15, RPS20, and RPS11;
a gene encoding a mitochondria protein, optionally selected from MT-001, MT-CO2, MT-ND4, MT-ND1, and MT-ND2;
a gene encoding an actin protein, optionally selected from ACTG1 and ACTB;
a gene encoding a eukaryotic translation factor, optionally selected from EEF1A1, EEF2, and EIF1;
a gene encoding a histone, such as H3F3A and H3F3B; or a gene selected from FTL, FTH1, TPT1, TMSB10, GAPDH, PTMA, GNB2L1, NACA, YBX1, NPM1, FAU, UBA52, HSP90AB1, MYL6, SERF2, and SRP14.

7. The genetically modified mammalian cell of claim 3, wherein the STEL is a GAPDH gene.

8. The genetically modified mammalian cell of claim 3, wherein the STEL is a ribosomal protein gene.

9. The genetically modified mammalian cell of claim 8, wherein the STEL is a ribosomal protein L (RPL) gene, optionally selected from RPL13A, RPL7, and RPLP0 genes.

10. The genetically modified mammalian cell of claim 1, wherein the cell is a pluripotent stem cell (PSC).

11. The genetically modified mammalian cell of claim 10, wherein the PSC is a human embryonic stem cell (ESC) or a human induced PSC (iPSC).

12. The genetically modified mammalian cell of claim 1, wherein the cell is a differentiated cell.

13. The genetically modified mammalian cell of claim 12, wherein the differentiated cell is derived from a human PSC, optionally selected from a human ESC and a human iPSC.

14. The genetically modified mammalian cell of claim 12, wherein the differentiated cell is
a human immune cell, optionally selected from a T cell, a T cell expressing a chimeric antigen receptor (CAR), a suppressive T cell, a myeloid cell, a dendritic cell, and an immunosuppressive macrophage;
a cell in the human nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron;
a cell in the human cardiovascular system, optionally selected from a cardiomyocyte, an endothelial cell, and a nodal cell;
a cell in the human metabolic system, optionally selected from a hepatocyte, a cholangiocyte, and a pancreatic beta cell, or
a cell in the human ocular system, optionally selected from a retinal pigment epithelial cell, a photoreceptor cone cell, a photoreceptor rod cell, a bipolar cell, and a ganglion cell.

15. The genetically modified mammalian cell of any one of the preceding claims claim 1, wherein the transgene is inserted into the 3′ untranslated region of the gene locus.

16. The genetically modified mammalian cell of claim 1, wherein the transgene sequence is linked in frame to the STEL gene sequence through a coding sequence for a self-cleaving peptide, or linked to the STEL gene sequence through an internal ribosomal entry site (IBES).

17. The genetically modified mammalian cell of claim 1, wherein the transgene encodes a therapeutic protein, an immunomodulatory protein, a reporter protein, or a safety switch signal.

18. The genetically modified mammalian cell of claim 1, wherein the genome of the cell further comprises an exogenous suicide gene, optionally in a STEL locus in the genome, wherein the exogenous suicide gene, once activated, causes apoptosis of the cell.

19. The genetically modified mammalian cell of claim 18, wherein the suicide gene is Herpes simplex virus (HSV) thymidine kinase (TK) gene.

20. A pharmaceutical composition comprising the genetically modified mammalian cell of claim 1 and a pharmaceutically acceptable carrier.

21. A method of treating a human patient in need thereof, comprising introducing the genetically modified mammalian cell of claim 1 to the patient, wherein the mammalian cell is a human cell.

22. The method of claim 21, wherein the human patient is in need of graft transplantation, or has inflammation, optionally neuroinflammation, an autoimmune disease, or cancer.

23-24. (canceled)

25. A method of generating the genetically modified mammalian cell claim 1, comprising
providing a cultured mammalian cell, and
introducing said transgene into a STEL site in the genome of the cultured cell.

26. The method of claim 25, wherein the introducing step is performed through CRISPR gene editing.
